(19) United States
(12) Patent Application Publication
(10) Pub. No.: US 2025/0288773 A1
(43) Pub. Date: Sep. 18, 2025

(54) Artificially Intelligent Systems, Methods and Media for Techniques Utilized to Access Subconscious and Unconscious States and Cause Behavioral Change

(71) Applicant: Burble, Inc., Philadelphia, PA (US)

(72) Inventors: Daniel C. Taylor, Philadelphia, PA (US); William Tustin, Yardly, PA (US); Alexandra Janelli, Philadelphia, PA (US)

(21) Appl. No.: 19/076,101

(22) Filed: Mar. 11, 2025

Related U.S. Application Data
(60) Provisional application No. 63/565,477, filed on Mar. 14, 2024.

(51) Int. Cl. A61M 21/02; A61B 5/00

(52) U.S. Cl. CPC A61M 21/02; A61B 5/7267

(57) ABSTRACT

Exemplary embodiments include a computer-implemented method of training a neural network and/or a large language model for automatically collecting, analyzing, and transmitting data to induce a mental state and change a behavior in a mammal.

### CROSS REFERENCE TO RELATED APPLICATIONS

This application claims the priority benefit of U.S. Provisional Patent Application Ser. No. 63/565,477 filed on Mar. 14, 2024 and titled, “Artificially Intelligent Systems, Methods and Media for Techniques Utilized to Access Subconscious and Unconscious States and Cause Behavioral Change,” the entirety of which is incorporated by reference herein.”

### FIELD OF INVENTION

The present technologies pertain to systems, methods and media for employing artificial intelligence for inducing a mental state and causing an emotional, physical or behavioral change in a mammal.

### SUMMARY OF EXEMPLARY EMBODIMENTS

Exemplary systems, methods and media (also known as Burble™) include an artificially intelligent (“AI”) driven personalized script generation that uses a Large Language Model (“LLM”), tiny language model and/or Neural Network (“NN”) to create personalized techniques utilized to access subconscious and unconscious states, guided visualization, meditation or other subconscious state(s) or relaxation inducing scripts based on user inputs and/or selections. A rapid voice synthesis process converts scripts into audio using a voice synthesizer or generator. Additionally, a user may pick the sound of the voice (e.g., male or female, accent or no accent, along with selecting a language). In other embodiments, a human voice recording without the use of AI may be employed. A user experience workflow customizes user experiences, including selecting issues to address, music and/or other audio and video and setting a session's conclusion. Multisensory elements integrate audio with AI-generated content to enhance the user experience in a wellness, mental health, meditation, relaxation or guided visualization context and/or a variety of other contexts.

Wellness, according to various exemplary embodiments, may include:
1. Active pursuit: Unlike simply the absence of illness, wellness is an active process. It involves conscious choices and actions directed towards achieving a state of holistic health and fulfillment.
2. Holistic perspective: Wellness goes beyond just physical well-being. It encompasses a multidimensional approach that integrates various aspects of life, including:

Physical: Maintaining a healthy body through proper nutrition, exercise, and preventive healthcare.

Mental: Cultivating emotional well-being, managing stress, and fostering positive mental health.

Social: Building and nurturing healthy relationships, fostering a sense of belonging, and engaging in meaningful social interactions.

Intellectual: Engaging in activities that stimulate the mind, encourage learning, and promote personal growth.

Spiritual: Finding meaning and purpose in life, connecting with a sense of transcendence, and aligning with personal values.

Environmental: Living in harmony with the environment, making eco-conscious choices, and engaging with nature.

Financial: Achieving financial security, managing resources effectively, and reducing financial stress.
3. Individualized journey: What constitutes “wellness” is unique to each individual. It depends on personal values, life circumstances, and goals.
4. Dynamic process: Wellness is not a static state; it is a continuous journey of growth and development. It can fluctuate throughout a person's life, requiring ongoing effort and adjustments.

Therefore, “wellness” can be understood as the active pursuit of a holistic and fulfilling life, encompassing physical, mental, social, intellectual, spiritual, environmental, and financial well-being, tailored to an individual's unique journey and evolving needs.

A system architecture for AI-driven wellness includes the way user inputs are processed, the way scripts are generated, audio is synthesized, video is created and content is delivered. Also included in some exemplary embodiments is an interactive tile-based selection system interface and real-time customization algorithm that enables real-time customization of the wellness experience based on user choices.

According to various exemplary embodiments, burble leverages cutting-edge artificial intelligence (AI) to revolutionize wellness experiences, offering users personalized content that adapts to their unique lifestyle and wellness goals. By analyzing user interactions, including user interactions with wearables and biometric data from wearable devices, burble's AI algorithms dynamically generate tailored wellness and hypnotherapy sessions. This innovative approach ensures that each user receives support specifically aligned with their personal challenges and preferences, enhancing the effectiveness of wellness interventions. The system's ability to learn and evolve with the user ensures a continually refined and deeply personal wellness journey.

In various exemplary embodiments, when a wearable device is utilized (e.g., EEG device or iWatch) an assessment that learns about the user (e.g., introvert/extrovert, things that help them relax, things that trigger them, what type of learner they are [visual, auditory, sensation-based, etc.]) in order to feed the user real-time generator audio, and video when desired, is monitored with the EEG. If it is not helping the user to relax or go into the desired wavelength, the custom content of audio and or video or both may be automatically adjusted until it does influence the user into the desired brainwave state in order to then push the subconscious/unconscious suggestions. Alternatively, some or all of these techniques may be used to bring a user to a lighter state in the event they have gone too deep or too asleep. Different depths are beneficial for different suggestions and come with different levels of amnesia coming out of the user despite the user at the time of the audio and or video content being delivered is 100% aware of what is happening.

In further exemplary embodiments, a burble may be automatically generated by the AI for a user based on what the AI has learned about the user.

According to various exemplary embodiments, burble is also designed for white label distribution, enabling partners to offer burble under their own branding while leveraging the burble technology. This feature is particularly beneficial in environments like universities, where burble can be customized with the institution's branding to address student wellness comprehensively, using data for ongoing enhancement. Another example would be an airline could integrate burble into its in-flight entertainment system, providing passengers with customized wellness sessions customized with the airline's brand and logos. This usage not only enhances the passenger experience but also aligns with the airline's commitment to customer well-being, all while maintaining the core functionalities and benefits of burble, powered by burble's technology.

The result: superior experiences, accessing the subconscious and unconscious states, while producing a shift in emotional, mental, physical and behavior change.

### BRIEF DESCRIPTION OF THE FIGURES

Certain embodiments of the present technology are illustrated by the accompanying figures. It will be understood that the technology is not necessarily limited to the particular embodiments illustrated herein.

FIG. 1 Shows an exemplary screenshot of the burble app.

FIG. 2 Shows an exemplary screenshot of the process of using the burble app.

FIG. 3 Shows an exemplary large language model.

FIG. 4 Shows an exemplary deep neural network.

FIG. 5 Shows an exemplary screenshot for initiating the burble process.

FIG. 6 Shows an exemplary screenshot for selecting a treatment in the burble process.

FIG. 7 Shows an exemplary screenshot for selecting an area to focus on within the treatment in the burble process.

FIG. 8 Shows an exemplary screenshot for selecting a track in the area to focus on within the treatment in the burble process.

FIG. 9 Shows an exemplary screenshot for selecting an induction in the area to focus on within the treatment in the burble process.

FIG. 10 Shows another exemplary screenshot for selecting an induction in the area to focus on within the treatment in the burble process.

FIG. 11 Shows an exemplary screenshot for selecting music in the burble process.

FIG. 12 Shows an exemplary screenshot for selecting an ending in the burble process.

FIG. 13 Shows an exemplary screenshot for generating a burble in the burble process.

FIG. 14 Shows an exemplary screenshot for providing user feedback while the burble is generating in the burble process.

FIG. 15 Shows an exemplary screenshot for playing a burble in the burble process.

FIG. 16 Shows an exemplary screenshot for an overview of the burble process.

FIGS. 17A through 17Q Show a customizable dashboard for burble.

### DETAILED DESCRIPTION

Exemplary embodiments include a networked (e.g., internet) computer-implemented method of training a neural network and/or a large language model for automatically collecting, analyzing, and transmitting data to induce a mental state and change a behavior in a mammal, the method including collecting a first set of data relevant to automatically collecting, analyzing, and transmitting data to induce the mental state and change a behavior in a mammal, applying one or more transformations to the collected first set of data to create a first modified set of data, creating a first training set comprising the first collected set of data, the first modified set of data and a first set of non-transformed data, training the neural network and/or large language model in a first stage using the first training set, creating a second training set for a second stage of training comprising the first training set and the first set of non-transformed data that are incorrectly transformed after the first stage of training, and training the neural network and/or large language model in a second stage using the second training set to automatically collect, analyze, and transmit data to induce a mental state and change a behavior in a mammal.

In various exemplary embodiments, burble is connected to a network, but is also being designed to function offline, allowing users access to its features without an internet connection. This offline capability is especially beneficial in scenarios like air travel, where WIFI may not be available. By localizing burble's content, burble ensures that passengers can enjoy the benefits of burble, enhancing their flying experience with accessible wellness and relaxation tools, regardless of their connectivity status.

Mammals may include humans including children, domestic animals, wild animals, livestock, zoo animals and the like. In some situations, the assistance of another party might be required to input and receive information.

Data may be received via keyboard, keypad, touchscreen, mobile device touchscreen and the like. Data may be inputted and/or received via wired and/or wireless networks. Data may be inputted and/or received by voice and/or audio and may involve the use natural language processing and/or voice recognition. Data may be inputted and/or received from sensors, wearables and/or devices including but not limited to:

### 1. Fitness Trackers and Smartwatches:

Information provided: Heart rate, steps taken, distance traveled, calories burned, sleep patterns, and some models can track blood oxygen saturation (SpO2) and skin temperature.

Benefits: Convenient and affordable way to monitor basic health metrics and encourage physical activity.

### 2. Smart Rings:

Information provided: Similar to fitness trackers, but some models can also track heart rate variability (HRV), a more advanced metric of heart health.

Benefits: Discreet and comfortable to wear, may offer additional health insights through HRV tracking.

### 3. Blood Pressure Monitors:

Information provided: Systolic and diastolic blood pressure, some models can also detect irregular heartbeats.

Benefits: Important for monitoring blood pressure, especially for individuals with hypertension or at risk of developing it.

### 4. Blood Glucose Monitors:

Information provided: Blood sugar levels, some models offer continuous glucose monitoring (CGM) for real-time tracking.

Benefits: Essential for managing diabetes and maintaining healthy blood sugar levels.

### 5. Smart Scales:

Information provided: Weight, body mass index (BMI), body fat percentage, muscle mass, and some models can track bone density and water weight.

Benefits: Provide a more comprehensive picture of body composition and health trends.

### 6. Electrocardiogram (ECG) Monitors:

Information provided: Electrical activity of the heart, can detect arrhythmias and other heart rhythm irregularities.

Benefits: Valuable for diagnosing and monitoring heart conditions.

### 7. Pulse Oximeters:

Information provided: Blood oxygen saturation (SpO2), heart rate, some models can track perfusion index (PI) which reflects blood flow.

Benefits: Useful for monitoring oxygen levels, especially for individuals with respiratory conditions or sleep apnea.

### 8. Smart Thermometers:

Information provided: Body temperature, some models can track trends and integrate with health apps.

Benefits: Convenient and accurate way to monitor temperature, especially for children or individuals prone to infections.

### 9. Sleep Trackers:

Information provided: Sleep duration, sleep stages (light, deep, REM), sleep quality, some models can detect snoring and other sleep disturbances.

Benefits: Help identify and improve sleep patterns, promoting better overall health and well-being.

### 10. Brainwave Monitors:

Information provided: Brainwave activity (EEG), some models can track stress levels, focus, brainwaves, and relaxation.

Benefits: Emerging technology with potential applications in stress management, meditation, and neurofeedback training. Brainwave monitoring can help the AI system determine the subconscious state of the user and provide generated scripts to help the user go into a different brainwave state in order to access the subconscious or unconscious state in order to create emotion, behavioral physical or mental states.

11. Fingerprint scanners: These devices measure the unique pattern of ridges and valleys on a person's finger. They provide high accuracy and are widely used for security purposes.

12. Iris scanners: These devices measure the unique pattern of the iris, the colored ring around the pupil of the eye. They offer high accuracy and are less susceptible to forgery than fingerprints.

13. Facial recognition systems: These systems use cameras and software to analyze the unique features of a person's face, such as the distance between their eyes, the shape of their nose, and the contours of their jawline. They are becoming increasingly sophisticated and are used for various applications, including security, identification, and marketing.

14. Voice recognition systems: These systems use microphones and software to analyze the unique characteristics of a person's voice, such as their pitch, timbre, and pronunciation. They are commonly used for voice authentication, access control, and dictation.

15. Retinal scanners: These devices measure the unique pattern of blood vessels in the retina of the eye. They offer high accuracy but require specialized equipment and are less commonly used than other biometric technologies.

Eye tracking and retina tracking may revolutionize personalized wellness in several ways:

### 1. Identifying Emotional States and Stress Levels:

Eye movements and pupil dilation are linked to emotional states. AI algorithms can analyze eye tracking data to detect subtle changes in pupil size, blink rate, and gaze patterns, potentially indicating:

Stress: Increased pupil dilation, faster blink rate, and erratic saccadic eye movements (rapid shifts in gaze) can be associated with stress.

Focus: Sustained attention might be reflected in reduced blink rates and fixated gaze patterns.

Relaxation: Slower blink rates and smoother eye movements could indicate a relaxed state.

### 2. Personalized Monitoring and Tracking:

AI can analyze eye tracking data over time to identify individual baselines and deviations for various emotional states. This allows for:

Personalized stress management: Identifying individual stress triggers through recurring patterns in eye movements.

Mood tracking and monitoring: Tracking changes in emotional states over time to understand personal patterns and potential triggers for emotional shifts.

Evaluating the effectiveness of interventions: Assessing the impact of wellness practices like meditation or breathing exercises on individual emotional responses.

### 3. Enhanced Mental Health Interventions:

Eye tracking data, combined with other physiological data (e.g., heart rate, skin conductance), can be used by AI to:

Personalize therapeutic interventions: Tailor interventions like cognitive behavioral therapy (CBT) or mindfulness exercises to specific needs and emotional responses identified through eye tracking.

Develop real-time feedback mechanisms: Provide real-time feedback during therapy sessions, guiding individuals towards calmer emotional states based on their eye movements.

Monitor treatment progress: Track progress in managing emotional states through changes in eye movement patterns over time.

### 4. Improved Human-Computer Interaction:

Eye tracking can be integrated into digital wellness platforms to:

Enhance user experience: Adapt the interface and content based on user attention and engagement, measured through eye movements.

Optimize learning and educational experiences: Personalize learning materials and adjust the pace based on individual focus levels indicated by eye movements.

Detect fatigue or distraction: Identify signs of fatigue or distraction through eye movements and provide alerts or adjust content difficulty accordingly.

### 5. Early Detection of Potential Health Issues:

There may be potential links between specific eye movement patterns and certain neurological conditions. AI analysis of eye movements, in conjunction with other diagnostic tools, might offer:

Early detection of potential cognitive decline: Identifying subtle changes in eye movements that may be associated with early stages of cognitive decline and support for diagnosing specific conditions like ADHD, autism spectrum disorder, or concussions by analyzing eye movement patterns.

16. Hand geometry scanners: These devices measure the size and shape of a person's hand. They are less accurate than fingerprint or iris scanners but are still used in some applications, such as time and attendance tracking.

17. Gait recognition systems: These systems use cameras and software to analyze the way a person walks, including their stride length, leg swing, and posture. They are still under development but have the potential to be used for security and surveillance applications in addition to health and balance issues.

18. Ear geometry scanners: These devices measure the size and shape of a person's ear. They are not as widely used as other biometric technologies but may be used in some security applications.

19. Odor recognition systems: These systems use sensors to analyze the unique scent of a person. They are still in the early stages of development but have the potential to be used for security and identification applications as well as aromatherapy.

Other data inputted and/or received may include DNA information, RNA (including messenger RNA) information, information from blood and/or urine testing, breathe analysis data, image data (e.g. Xray, MRI, CT) and the like.

Mental states may include:

### 1. Emotional States:

Positive: May included but not limited to, joy, love, contentment, excitement, gratitude, peace.

Negative: May included but not limited to, fear, anger, sadness, frustration, anxiety, boredom

Neutral: May included but not limited to, curiosity, surprise, confusion, acceptance, indifference

### 2. Cognitive States:

Focused: May included but not limited to, attention, concentration, alertness, mindfulness.

Unfocused: May included but not limited to, daydreaming, distraction, drowsiness, fatigue.

Problem-solving: May included but not limited to, critical thinking, creativity, analysis, decision-making.

Learning: May included but not limited to, memory recall, comprehension, understanding, insight.

### 3. Physiological States:

Relaxed: May included but not limited to, calmness, peacefulness, ease, muscle relaxation.

Stressed: May included but not limited to, tension, anxiety, agitation, muscle tension, increased heart rate.

Tired: May included but not limited to, fatigue, drowsiness, sleepiness, lack of energy.

Energetic: May included but not limited to, alertness, enthusiasm, vigor, motivation.

### 4. Altered States of Consciousness:

Techniques utilized to access subconscious and unconscious states: May included but not limited to, deep relaxation, focused attention, heightened suggestibility.

Meditation: May included but not limited to, tranquil awareness, reduced reactivity, inner peace.

Sleep: May included but not limited to, dreaming, unconsciousness, restorative processes.

Drug-induced (and/or other substance-induced) states: May included but not limited to, hallucination, euphoria, altered perception, impaired judgment.

Contrary to popular belief, hypnotizing someone isn't about controlling their mind or making them do things against their will. Instead, it involves guiding them into a state of deep relaxation and focused attention, where they are more open to suggestion and suggestion-based change.

To access the conscious or unconscious and relaxed states associated with meditation and other practices, techniques utilized to access subconscious and unconscious states, guided visualization, and other practices someone responsibly and ethically the following is needed:

Willingness and Consent: The most important element is the individual's willingness and informed consent to be a participant in the process. They should understand what the process is, what it might involve, and have the freedom to stop at any time.

Relaxation Techniques: The practitioner typically uses various relaxation techniques like guided imagery, deep breathing exercises, visualizations, Neurolinguistic Programming, neuroplasticity techniques, systematic desensitization techniques, inner child work, reframing of belief systems, repetitive suggestions formulated in a variety of direct, metaphorical and other means, and other body and mental relaxation enhancing techniques to help the person reach a state of focused attention, deep relaxation in the body, and alpha, beta, theta or deep brainwave lengths. This state is similar to being deeply absorbed in a book or daydreaming, but with a deep relaxation of the body and acute awareness of the practitioners voice.

Suggestions: Once relaxed and focused, the practitioner offers suggestions, directives, choices, open ended questions for consideration aimed at helping the individual achieve their goal, whether it is quitting smoking, overcoming anxiety, improving sleep, or any other emotional, physical, or behavioral association that they are looking to change. These suggestions are tailored to the individual and worded in a manner that is most susceptible to the user, metaphorically or directly, and calmly. These suggestions offer the user a basis to release blocks and subconscious accept new ways of behaving, thinking or feeling.

Susceptibility varies: Some people are more naturally susceptible to techniques utilized to access subconscious and unconscious states, guided visualization, meditation and other practices that allow access to the subconscious or conscious mind than others, but everyone has the potential to experience some level of the subconscious and unconscious state. These states are naturally accessed when we fall asleep at night and wake up in the morning.

Because of the above factors, the use of AI as described herein results in superior subconscious and unconscious access and behavior, physical, mental or emotional change.

Behavior or behavioral changes may include, but is not limited to (otherwise known as the library, i.e., Nos. 1-4 below):

### 1. Physical Health:

Diet: Eating less to lose weight, consuming more fruits and vegetables, reducing sugar or processed foods, increasing whole grains, adopting specific dietary plans, being in control of food or cravings, better relationships with food, fueling the body properly with the right foods to support health or dietary needs in order to suppress any existing conditions (for example: celiacs. Helping users feel confident in their choice to avoid it.

Exercise: Starting or increasing physical activity, joining a gym, participating in specific sports, incorporating more movement into daily routines, feeling more comfortable in the gym, enjoyment of moving the body, not comparing to others, . . .

Sleep: Establishing regular sleep schedules, developing bedtime routines, improving sleep hygiene, seeking professional help for sleep disorders, changing the negative associations with sleep, letting go of past sleep patterns, shifting the limiting beliefs about sleep and/or identification as having a specific condition, such as being an “insomniac”.

Substance Use: Quitting smoking or alcohol, reducing caffeine intake, managing drug dependencies through professional support.

### 2. Mental and Emotional Wellbeing:

Stress Management: Practicing relaxation techniques like meditation or yoga, engaging in hobbies and activities they enjoy, managing time effectively, seeking professional help for stress management. It can also include changing the users relationship with stress, while also shortening the stress response time and providing tools to move through it more effectively.

Negative Thought Patterns: Challenging negative thoughts and self-talk, practicing gratitude and positive affirmations, developing cognitive behavioral therapy (CBT) skills while helping to self-identify as a confident and more grounded and centered individual.

Relationship Building: Improving communication skills, practicing active listening, expressing emotions constructively, setting healthy boundaries, seeking professional relationship counseling, while also helping build inner self-confidence through a variety of suggestions to the subconscious and unconscious mind.

Coping with Mental Health Conditions: Implementing therapy recommendations, managing medications effectively, joining support groups, building a supportive network.

### 3. Personal Growth and Development:

Learning New Skills: Taking courses, reading books, participating in workshops, practicing new skills consistently. Feeling more confident or free with a willingness to try new things without fear.

Time Management: Setting goals, prioritizing tasks, developing routines, limiting distractions, utilizing time management tools, setting better sleep schedules and routines while also creating better relationships with the idea of time and how they interact with time and the allocation of it.

Developing Habits: Implementing positive habits like journaling, exercising regularly, cleaning regularly, setting achievable goals.

Breaking Bad Habits: Identifying triggers and their underlying association, coping mechanisms that no longer serve a purpose for growth and development replacing bad habits with positive ones, seeking professional help if needed.

Financial Management: Budgeting, saving money, paying off debt, investing wisely, seeking financial planning advice, creating a new association and relationship with money and/or working.

### 4. Social and Environmental:

Reducing Environmental Impact: Conserving energy, recycling, using sustainable products, reducing waste, supporting eco-friendly businesses.

Volunteering and Community Involvement: Donating time and resources to causes they care about, participating in community service projects, building stronger relationships with their community.

Empathy and Compassion: Practicing active listening, showing understanding towards others, offering help and support, engaging in acts of kindness.

Social Skills Development: Initiating conversations, building rapport, expressing themselves effectively, resolving conflicts constructively.

The computer-implemented method of training a neural network and/or a large language model for automatically collecting, analyzing, and transmitting data to induce a mental state and change a behavior in a mammal and the steps of:
1. Collecting a first set of data;
2. Applying one or more transformations to the collected first set of data to create a first modified set of data;
3. Creating a first training set comprising the first collected set of data, the first modified set of data and a first set of non-transformed data;
4. Training the neural network and/or large language model in a first stage using the first training set;
5. Creating a second training set for a second stage of training comprising the first training set and the first set of non-transformed data that are incorrectly transformed after the first stage of training; and
6. training the neural network and/or large language model in a second stage using the second training set to automatically collect, analyze, and transmit data to induce a mental state and change a behavior in a mammal, are significant.

Inducing a mental state and changing a behavior is a complex process requiring robust AI. Like with using AI for facial recognition, there can be a huge variability in the data that is being processed and a limited amount of data for training the AI. Accordingly, according to various exemplary embodiments, burble can expand the training set by applying mathematical transformation functions on an acquired set of data. For example (and not by limitation), burble can determine a statistical standard deviation within a particular data set and apply increasing or decreasing increments of the standard deviation to the data set. The AI can be trained with this expanded training set using stochastic learning with backpropagation which is a type of machine learning algorithm that uses the gradient of a mathematical loss function to adjust the weights of an AI network. Unfortunately, the introduction of an expanded training set tends to increase false positives when classifying data. Accordingly, the second feature is the minimization of these false positives by performing an iterative training algorithm, in which the AI network is retrained with an updated training set including the false positives. This combination of features provides a robust AI network for inducing a mental state and changing a behavior.

### Burble.

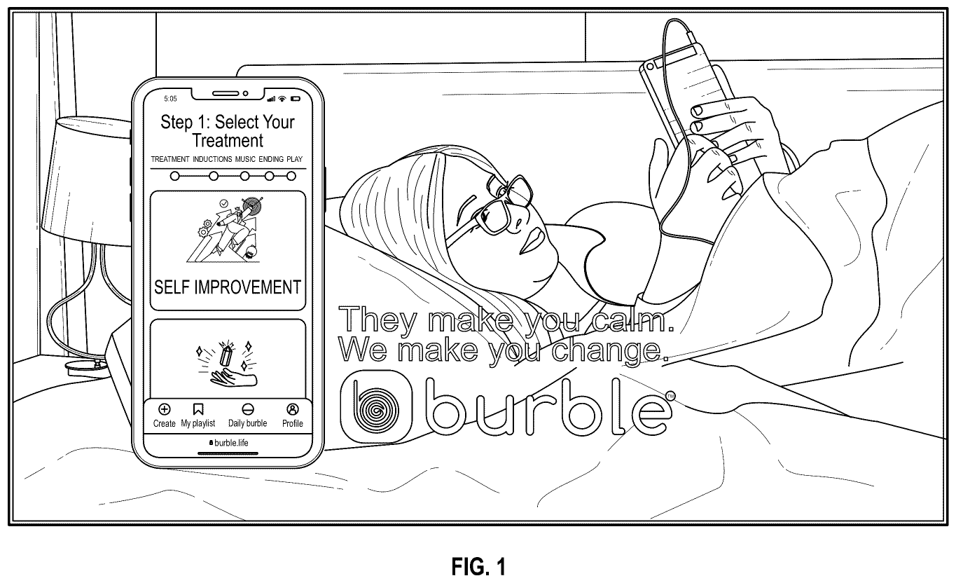

FIG. 1 Shows an exemplary screenshot of the burble app.

As shown in FIG. 1, burble changes behavior.

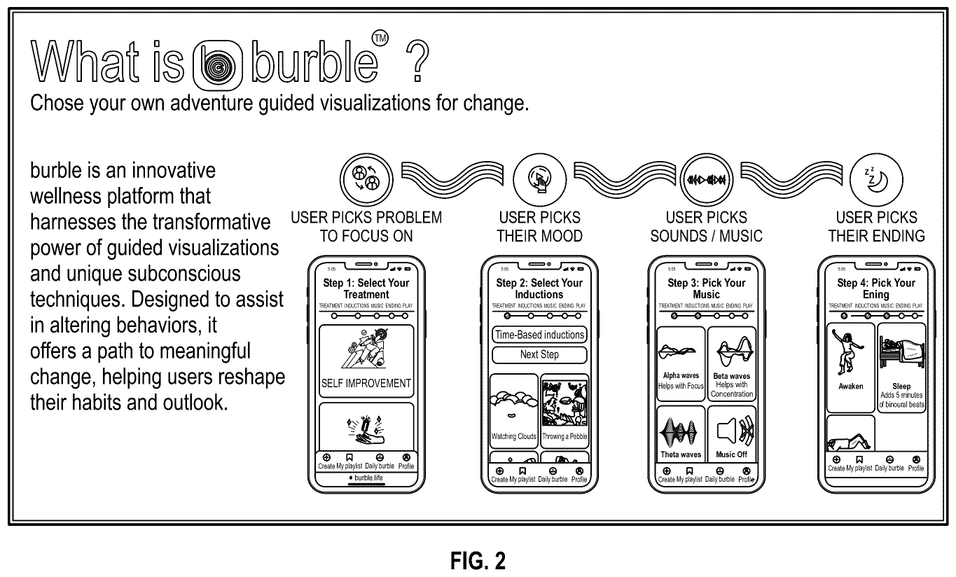

FIG. 2 Shows an exemplary screenshot of the process of using the burble app.

As shown in FIG. 2, the user picks a problem to focus on, picks their mood, picks their sounds/music and picks their ending.

Personalization in wellness programs is crucial for engagement and effectiveness, offering clear benefits such as better motivation, knowledge, engagement, and health and mental health outcomes. A personalized approach ensures that wellness interventions are relevant and meaningful to each user, significantly enhancing their effectiveness and user satisfaction.

During the COVID-19 pandemic, the use of health and wellness apps saw a significant increase, highlighting the role these technologies played in supporting healthy behaviors and mental well-being.

The popularity of health and wellness apps, including those focused on mental health, surged during the pandemic. This increase in app downloads and usage underscores the growing demand for digital health solutions that offer support for physical activity, diet, sleep, and mental health management.

The exemplary embodiments described herein are positioned to solve the problem of providing highly personalized and relevant, engaging, accessible, and efficient wellness support that dynamically responds to the unique needs of each user. This approach not only enhances the user experience and outcome but also broadens the reach and impact of wellness interventions across diverse populations.

The exemplary embodiments described herein address a gap in the wellness and self-help app market by solving several interconnected problems:
1 Lack of Personalization: Many existing wellness platforms offer static, one-size-fits-all content that may not meet the individual needs, preferences, or current emotional or physical state of each user. Exemplary embodiments solve this by using AI to generate dynamic, personalized content that adapts in real-time to a user's feedback and biometric data, offering a more tailored and effective wellness experience.
2. Limited Engagement: Users may lose interest in wellness apps when the content becomes repetitive or fails to evolve with their changing needs. The exemplary embodiments described herein keep users engaged with fresh, customized content designed to match their personal growth and varying wellness goals, thereby potentially increasing long-term engagement and satisfaction. Also, existing solutions may not adequately serve non-English speakers or those seeking content that reflects their cultural background or specific wellness concerns. By leveraging AI, exemplary embodiments have the potential to offer a broader range of languages and culturally relevant content, making wellness more accessible and inclusive.
3. Static Content: Many apps rely on static libraries of pre-recorded content that can become repetitive over time, limiting long-term user engagement and effectiveness. The dynamic content provided by exemplary embodiments ensure that users always have access to fresh, relevant content that evolves with their changing needs and goals.
4. Accessibility and Inclusivity: Traditional wellness apps may not cater to the diverse linguistic and cultural backgrounds of a global user base. By leveraging AI for content creation, exemplary embodiments have the potential to offer a wider range of languages and culturally relevant content, making wellness more accessible and inclusive.
5. Integration with Wearable Technology: While some wellness apps incorporate data from wearable devices, they may not fully exploit this data to offer real-time, context-sensitive interventions. Exemplary embodiments use of biometric, brainwave and other mental, physical and emotional markers data from wearables to inform and adjust content delivery represents a significant advancement, providing users with immediate support exactly when they need it, such as stress relief prompts based on, for example, but not limited to, detected increases in heart rate or change in breathing rates.
6. Cost and Resource Efficiency in Content Production: Producing high-quality, diverse wellness content traditionally requires significant investments in human resources, including experts for content development and voice actors for recordings, as well as technical resources for recording and editing. The AI-driven content creation method described herein addresses these challenges by reducing the time, cost, and resources needed to produce and update extensive libraries of wellness content.
7. Real-Time Support Lacking: Users facing immediate stress, anxiety, or other wellness challenges may not find the instant support they need with pre-recorded content. Integration with wearable technology as described herein allows for real-time detection of stress indicators (like elevated heart rate or breathing changes), offering immediate, customized interventions to help users manage their state effectively. The herein described approach to using AI for creating dynamic and personalized content directly addresses these identified needs and trends. By focusing on customization and leveraging biometric data for tailored wellness interventions, personalized, effective, and accessible wellness solutions that resonate with users on an individual level may be provided.

AI focused on using techniques utilized to access subconscious and unconscious states, meditation, guided visualization and other wellness techniques to deliver robust, dynamically built custom audio to address undesired behaviors for a user may be provided. Exemplary embodiments may include dynamically generated video content, as well as other content and physical feedback, such as vibrations through a wearable device, and capturing biometric and other physiological data to further build and deliver real time dynamic content.

Exemplary embodiments may be used by individuals to, for example, improve their overall well-being and can also be used by healthcare providers to monitor and improve the well-being of their patients. Exemplary embodiments can be integrated into a variety of personal wellness devices, including wearable devices, mobile devices, and smart home devices.

The use of natural language processing techniques by the exemplary embodiments described herein analyzes a patient's responses to a series of questions, prompts and other saved data, such as spoken language preferences. They then generate content that is customized to address a user's specific concerns and goals.

Also included is a user interface that may allow a third party to input information about a user and any associated goals, treatment plans or similar plans in order to supplement and support their therapy. The platform can also present pre-made content scripts, assessments or recordings (or all) that help to create treatment plans and programs. Exemplary embodiments may also allow a user to input information and choose on screen prompts to create content. Based on these responses by a user, Exemplary embodiments may use AI to create customized content and programs that are tailored to a user's and/or patient's individual needs or goals.

By utilizing AI, exemplary embodiments may create dynamic, customized content in any spoken language that is tailored to the individual needs of each user. This can lead to more effective and better outcomes for the user. User intake assessment can also custom curate audio wellness programs that are based on the users presenting issues and also works to help the underlying causes of the behaviors the user is presenting. The AI works to help get to the underlying cause and associations on a subconscious level for the issues, not just the presenting issues. It can also help release any associated or stuck traumas in the unconscious mind. As an example, consider a user struggling with insomnia. The AI does not just address the symptom (which would be the inability to sleep); it delves deeper. Through a tailored series of questions, it uncovers underlying causes for the inability to sleep, such as performance anxiety or work-related stress, offering a targeted approach to improving sleep beyond surface-level solutions. This approach is what burble calls the dig down in order to find the root associations of presenting issues.
8. Other wellness offerings use human recorded audio, which limits their offerings in the following ways:

Efficiency and Flexibility: Traditional audio production is a resource-intensive process, involving lengthy scripting, recording, and editing phases. Not all audio sounds the same due relative human errors including vocal changes or other.

Cost-Effectiveness: The need for specialized recording equipment, studio space, and skilled audio engineers makes professional audio production expensive.

Scalability: The conventional process of audio recording is inherently limited by the capacity to produce new content.

Language and Accessibility: Traditional audio is confined by the language abilities of the voice talent, posing barriers to global accessibility.

Personalization: Human-recorded content offers a static, one-size-fits-all solution.

Length: One of the unique features described herein is flexibility in session duration, allowing users to customize the length of their wellness sessions according to their schedules and need. This adaptability ensures that even those with only a brief window of time can benefit from personalized support, setting the exemplary embodiments described herein apart from other wellness apps that offer only pre-recorded sessions of fixed length. This “choose-your-own-duration” approach addresses a critical barrier to consistent app use, as users are no longer constrained by the inability to fit a session into their busy lives. By enabling users to construct a session that fits their available time, the exemplary embodiments described herein significantly enhance the likelihood of consistent use, offering a more personalized and practical solution for everyday wellness.

Using AI to create dynamic audio offers several advantages over traditional human-based recording methods, significantly enhancing the flexibility, scalability, and personalization of audio content. By transcending the limitations of human-recorded audio, the technology disclosed herein sets a new standard for flexibility, accessibility, and personalization in wellness technology.

The AI-generated exemplary embodiments prove superior because of:

Scalability: AI can generate countless variations of audio content quickly and efficiently, allowing for a vast library of offerings without the bottleneck of human production capacity.

Personalization: AI technology can tailor audio content to the individual preferences and needs of users in real-time, offering a more personalized experience that can adapt to a user's mood, preferences, and progress.

Accessibility: With AI, it is easier to offer content in multiple languages and dialects, improving accessibility for a global audience without the need for multilingual speakers or translations, which can be costly and time-consuming.

Consistency: AI can maintain a consistent quality and tone across all audio content, ensuring a uniform user experience that is not affected by human variables like mood, health, or performance inconsistencies.

Innovation: AI can experiment with new sounds, tones, and musical elements, incorporating a wide range of therapeutic and non-therapeutic audio techniques that might be beyond the skill set of human producers, including, but not limited to, binaural beats or isochronic tones for enhanced mental states.

Both isochronic tones and binaural beats are methods used to influence brain waves and potentially alter mental states. However, they work in different ways:

### 1. Isochronic Tones:

Mechanism: These are single, pulsed tones that are played repeatedly at a specific frequency, creating a regular rhythm. The brain is thought to respond to this rhythm by entraining its own brain waves to match the frequency of the tones.

Influencing brain waves: Depending on the frequency of the tones, isochronic tones are believed to target different brain wave states:

Slower frequencies (below 10 Hz): May promote relaxation, meditation, and sleep.

Mid-range frequencies (10-20 Hz): May enhance focus, concentration, and learning.

Higher frequencies (above 20 Hz): May improve alertness and cognitive performance.

### 2. Binaural Beats:

Mechanism: These involve creating the perception of a beat by presenting two slightly different tones, one to each ear. The brain then processes the difference in frequencies as a single beat at the difference frequency between the two tones.

Influencing brain waves: Like isochronic tones, binaural beats are also believed to entrain brain waves to match the difference frequency:

Slower difference frequencies: May induce relaxation and sleep.

Mid-range difference frequencies: May enhance focus, concentration, and creativity.

Higher difference frequencies: May increase alertness and energy.

Here are a few applications:

### 1. Personalized Brainwave Entrainment:

AI algorithms can analyze an individual's brainwave activity through EEG (electroencephalogram) sensors or other non-invasive techniques.

Based on the analysis, the AI can recommend personalized isochronic tones or binaural beat frequencies tailored to their specific needs and desired mental state, such as promoting relaxation, focus, or sleep.

This personalized approach could potentially increase the effectiveness of these techniques compared to generic audio tracks.

### 2. Adaptive and Responsive Audio Experiences:

AI can continuously monitor an individual's physiological and emotional state through sensors and/or self-reported data.

Based on real-time data, the AI can dynamically adjust the isochronic tones or binaural beat frequencies to optimally support their desired mental state.

For example, if someone becomes stressed during meditation, the AI might adjust the audio frequency to promote deeper relaxation and calm them down.

### 3. Integrated Wellness Management Platforms:

AI can integrate isochronic tones and binaural beats with other wellness practices like meditation guidance, mindfulness exercises, and sleep monitoring.

These platforms can provide a holistic approach to well-being, offering users personalized recommendations and adjustments based on their individual goals and progress.

### 4. Self-regulation and Mental Health Support:

AI-driven applications incorporating isochronic tones and binaural beats could potentially offer support for managing stress, anxiety, and sleep disturbances.

### 5. Research and Development:

AI can analyze large datasets of brainwave activity and user feedback to improve the understanding of how isochronic tones and binaural beats affect different individuals and mental states.

This knowledge can be used to refine these techniques and develop more effective and personalized interventions for promoting well-being, including the blending AI-generated narrative content with user-selected music.

Cost Efficiency: Once developed, AI systems can produce audio content with minimal ongoing costs, significantly reducing the financial burden associated with studio time, equipment, and personnel required for human-based recordings.

Speed of Updates and Iterations: AI allows for rapid updates and iterations based on user feedback or emerging research. New content versions can be generated and deployed much faster than the traditional recording process would allow.

Integration of Data Insights: AI systems can integrate user data and insights to continuously improve and adapt the audio content for better outcomes, something that is not feasible with static, human-recorded tracks.

Environmental Sustainability: By eliminating the need for physical studio spaces and the associated energy consumption, AI-driven audio production is a more environmentally sustainable option.

Unlimited Creativity: AI can combine elements in novel ways that might not occur to human creators, potentially discovering new methods for promoting relaxation, focus, or wellness.

By leveraging AI for custom/personalized audio creation, the technologies described herein can push the boundaries of what is possible in wellness technology, offering a service that is not only innovative, but also deeply attuned to the evolving needs and preferences of its users.

The exemplary embodiments described herein provide an approach to wellness through AI-generated audio content marks a significant departure from traditional offerings in several key aspects:

Customization and Personalization: Unlike traditional methods that offer a one-size-fits-all solution, the technologies disclosed herein utilize AI to provide personalized audio experiences tailored to each user's specific needs, preferences, and wellness goals. This means that the content can adapt in real-time to the user's feedback or changes in their mental state, ensuring a more effective and engaging experience.

Scalability and Variety: Traditional wellness solutions are limited by the scope of their human-recorded content, which can be expensive and time-consuming to produce. In contrast, the AI-driven approaches disclosed herein enable the rapid creation of a vast and diverse library of audio content. This allows for a wider range of topics, techniques, and languages, making wellness accessible to a broader audience.

Cost-Effectiveness: The production of traditional audio content requires significant investment in recording equipment, studio space, and talent, including voice actors and audio engineers. The AI-generated audio as disclosed herein eliminates many of these costs, making it a more economically viable solution while still maintaining high-quality content delivery.

Rapid Content Development and Updates: Traditional content can quickly become outdated and may not respond to emerging wellness trends or user feedback without significant additional investment. The AI capabilities disclosed herein allow for quick content iteration and updates, ensuring the platform remains at the forefront of wellness technology.

Language and Accessibility: Traditional audio recordings are typically limited to the languages spoken by the available voice talent, which can restrict access for non-native speakers. The AI-generated audio disclosed herein can easily be produced in multiple languages, enhancing accessibility and inclusivity for users worldwide.

Innovative Techniques: The AI disclosed herein allows the exploration and incorporation of cutting-edge sound therapies and techniques that may be beyond the reach of traditional audio production, such as specific frequencies and sounds designed to enhance relaxation, focus, or other desired states.

Data-Driven Insights: The AI technology disclosed herein can leverage user data to continuously refine and improve its offerings based on real-world usage and outcomes. This contrasts with traditional methods, which lack the ability to dynamically adjust content based on user engagement and effectiveness. The AI-driven approach disclosed herein offers a more personalized, accessible, and flexible solution to wellness, leveraging technology to meet the evolving needs of users in ways that traditional human-based recordings cannot match. Additionally, burble has been trained on hundreds of recordings, papers, scripts, workshops, talks, lectures, podcasts, radio shows, interviews and more.

There are several additional ways the exemplary embodiments described herein may be expanded or applied that extend its impact across various domains:

Educational Tools: Utilizing the AI described herein to create personalized learning experiences, where audio content is tailored to the learning style and pace of each student. This could include language learning, meditation and focus techniques for better study habits, or even auditory learning modules for complex subjects.

Corporate Wellness Programs: Integrating the AI described herein into corporate wellness initiatives, offering employees customized audio sessions for stress reduction, productivity enhancement, and mental health support. This could be particularly beneficial in remote work environments, helping maintain a healthy work-life balance.

Healthcare and Therapy Support: Partnering with healthcare providers to offer supplementary therapy options, such as guided audio for pain management, pre- and post-surgery relaxation techniques, or support for mental health conditions like depression and anxiety, tailored to patient needs.

Fitness and Physical Well-being: Expanding into physical wellness, the technologies described herein could offer guided audio for yoga, meditation, or even personalized workout sessions, where the AI adjusts the pace and intensity based on the user's performance and feedback.

Sleep Aid Solutions: Developing specialized audio programs aimed at improving sleep quality, leveraging the AI described herein to adapt content based on the user's sleep patterns and preferences, such as guiding them through relaxation techniques or providing ambient soundscape adjustments in real-time.

Virtual Reality (VR) and Augmented Reality (AR) Integration: Merging audio content with VR and AR technologies to create immersive wellness experiences. This could include virtual meditation retreats or stress-relief scenarios, where the audio dynamically responds to the user's interactions within the virtual environment.

Cultural and Linguistic Adaptation: Using the AI described herein to not only translate but culturally adapt the audio content, making wellness practices accessible and relevant across different cultural contexts. This could involve incorporating region-specific wellness traditions and practices into the audio content.

Community and Social Well-being: Facilitating group wellness sessions where audio content is synchronized across multiple users, promoting social connections and support. This could be used in community centers, schools, or as part of public health initiatives.

Travel and Hospitality: Offering customized audio content for travelers and hotel guests, including relaxation audio for flights, personalized wellness content in hotel rooms, or guided audio tours with wellness components.

For example, in a “white label” example:

Biometric data is not a requirement for the user.

In some exemplary embodiments, a hotel guest would create an account and become a burble user, which would give burble access to their biometric data. Other hotel guests may see the burble offering while trying to “settle in” at their hotel and may try burble on a whim. Working with burble's “white label” partners, burble has captured their feedback and experiences, and then creates a general set of common complaints and issues for travelers and/or guests of the hotel.

Burble may provide a “general use” burble without all of the customization. For example:

Interactive Audio Books and Stories: Creating a new genre of interactive, wellness-focused audio books where the story and lessons adapt based on the listener's responses or state of mind, promoting mental health through storytelling.

These applications demonstrate the versatility of the AI-driven audio and/or video content disclosed herein, indicating that the technology may have far-reaching implications across various sectors, enriching lives through personalized wellness and learning experiences.

Integrating the exemplary embodiments described herein with wearables like an Apple iWatch to leverage biometric data opens up innovative pathways for personalized wellness interventions. This approach significantly enhances the technologies described herein to deliver real-time, customized content based on the user's physiological states.

For example, the Apple iWatch (as well as other devices) collects a variety of biometric data, categorized as:

### 1. Health and Fitness Tracking:

Heart Rate: The watch continuously monitors your heart rate, providing information about resting heart rate, heart rate variability, and real-time heart rate during workouts.

Blood Oxygen: The Apple Watch Series 6 and later models offer the ability to measure your blood oxygen levels, providing insights into your respiratory system and overall health.

Activity Monitoring: The watch tracks your daily steps, distance moved, and active calories burned, encouraging and motivating you to maintain an active lifestyle.

Workout Tracking: It offers various workout profiles like running, swimming, cycling, and strength training, recording workout duration, calories burned, and average pace.

Fall Detection: The watch can detect falls and automatically alert emergency services if needed.

### 2. Sleep Tracking:

Apple Watch Series 3 and later models can track your sleep patterns, providing insights into sleep duration, sleep quality, and sleep stages (deep sleep, light sleep, REM sleep).

### 3. Other Biometric Data:

Electrocardiogram (ECG) App: Apple Watch Series 4 and later models can generate an ECG (electrocardiogram) reading, providing insights into a person's heart rhythm and potentially detecting signs of atrial fibrillation.

Noise App: Introduced with WatchOS 9, the Noise app measures ambient noise levels and can notify a person if the noise levels reach potentially harmful levels, helping protect the person's hearing.

Here are some of the ways the exemplary embodiments described herein may be utilized, building on the example of using heart rate data for stress management:

Breathing Pattern Analysis: By monitoring a user's breathing rate, the technologies described herein may identify signs of anxiety or stress. In response, users may be alerted and provided with guided breathing exercises tailored to induce relaxation and improve the users' immediate well-being. This could be particularly useful during moments of acute stress or pre-event nerves, such as before a public speaking engagement.

Activity Level Adjustments: Using data on physical activity levels from a wearable device, the technologies described herein may recommend audio content aimed at balancing a user's activity and rest. For instance, after a period of prolonged inactivity, they might suggest a gentle movement or stretching session guided by audio, whereas after intense physical activity, they might recommend a relaxation or mindfulness session to aid in recovery.

Sleep Quality Insights: By analyzing sleep patterns, such as duration, consistency, and restfulness, the technologies described herein may offer personalized bedtime routines or relaxation techniques designed to improve sleep quality. This could include soothing soundscapes or guided visualizations tailored to a user's preferences and sleep data, helping to address issues like insomnia or irregular sleep schedules.

Mood Prediction and Regulation: Advanced algorithms including those described herein may use biometric data to predict a user's mood and emotional state, offering preemptive content designed to uplift, motivate, or calm. For example, a detected pattern of lethargy or low activity might trigger motivational audio content, whereas signs of agitation might lead to calming meditations.

Focus and Concentration Enhancement: By monitoring indicators of concentration, such as heart rate variability during tasks, the technologies described herein may identify moments when a user's focus is waning. It may then offer short, focused audio sessions designed to rejuvenate mental clarity and concentration, optimizing productivity and mental acuity.

Custom Wellness Journeys Based on Physiological Feedback: Over time, the technologies described herein may learn from a user's biometric feedback, tailoring longer-term wellness programs that adapt to the user's evolving needs. For example, if the data consistently shows elevated stress levels at certain times of day or week, exemplary embodiments may schedule preemptive relaxation sessions to mitigate this stress.

Integration with Health Goals: Users could set specific health-related goals within the exemplary embodiments described herein, such as reducing stress, improving cardiovascular health, or enhancing sleep quality. Exemplary embodiments may then use biometric data to track progress towards these goals, offering tailored content to support the user's journey and adjusting recommendations based on real-time data.

Another example: burble might detect that a person's heart rate and temperature increase significantly. Knowing this, burble may preempt and start getting the person to focus on something else around 12:50 pm every day to prepare.

This integration of wearables to aid in the creation of dynamic AI-driven content creation promises a highly adaptive, responsive, and personalized wellness experience. By leveraging real-time biometric data, the technologies described herein can more effectively support users in achieving a balanced state of mental and physical well-being, marking a significant advancement in personalized health and wellness technology.

For example, if a wearable transmits a body temperature of 88 degrees, AI may send a signal to a Nest thermostat to check the temperature of a user's room and increase it if it is low. Otherwise, burble may call an ambulance, family member and/or friend or the like.

Thus, there is a need for the AI-driven personalization, flexibility in session duration, and planned integration with biometric data for custom wellness experiences provided by and to be provided by the exemplary embodiments disclosed herein.

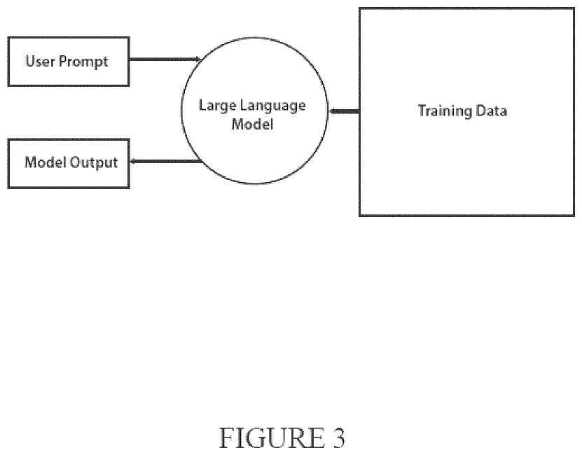

FIG. 3 shows an exemplary large language model. Shown in FIG. 3 is a user prompt, a large language model, training data, and a model output. A user prompt in a large language model (LLM) is a piece of text that is used to guide the LLM to generate a desired model output. The prompt can be used to specify the type of model output that the LLM should generate, as well as the style and tone of the output. The quality of the model output generated by an LLM is heavily influenced by the quality of the prompt. A well-crafted prompt will help the LLM to generate output that is more relevant, accurate, and creative.

A large language model (LLM) is a type of artificial intelligence (AI) model that is trained on a massive amount of text data. This data can be text from books, articles, websites, or any other source of text. The LLM learns the patterns and structure of the text data, and it can then use this knowledge to generate new text, translate languages, write different kinds of creative content, and answer questions in an informative way.

LLMs are advanced artificial intelligence algorithms trained on massive amounts of text data for the purposes of content generation, summarization, translation, classification, sentiment analysis and so much more. Smaller datasets are composed of tens of millions of parameters, while larger sets extend into hundreds of billions of data points. Depending on the purpose of the LLM, the training data will vary.

Example datasets and what their purposes include:

Social media posts: Publicly available social posts can be used to train the model to understand informal language, slang, and online trends, as well as to identify sentiment.

Academic papers: Scholarly articles can be used to understand terminology and technical language, as well as to extract key information.

Web pages: Publicly available web sites can be used to understand writing styles or increase the range of topics a large language model can understand.

Wikipedia: Because of the vast knowledge that Wikipedia houses, this can be used to increase the range of topics a large language model can understand.

Books: Books of various genres can be used to understand different writing styles, storyline development, and narrative structures.

Using the above examples, if a model is trained on social media posts and books, it becomes easier for the model to produce text in a human-like fashion because it has a clear understanding of formal and informal language. In reality, the answers it produces is highly dependent on the training data used.

Transformer architecture is the backbone of the transformer models like GPT and many other prominent LLMs. The transformer architecture is a neural network architecture that allows for parallel processing and is used by large language models to process data and generate contextually relevant responses. It consists of a series of layers, with each layer consisting of parallel processing components called attention mechanisms and feedforward networks. The attention mechanisms weigh the importance of each word, using statistical models to learn the relationships between words and their meanings. This allows LLMs to process sequences in parallel and generate contextually relevant responses. Still, models are trained for specific purposes so there is not a single model that does everything, including the exemplary AI described herein.

Large language models can process and understand human language at scale. These models use deep learning techniques to analyze vast amounts of text data, making them highly proficient in language processing tasks such as text generation, summarization, translation, and sentiment analysis.

One way to embrace large language models without incurring significant model development costs is through the integration of an AI copilot.

LLMs also can analyze and understand large amounts of data and information, allowing them to provide insightful recommendations to improve business processes and decision-making. Moreover, the conversational interface of LLMs makes it easier for teams to share and collaborate on ideas and projects, increasing productivity and streamlining the creative process.

Understanding large language model nuances is crucial in using them in real-world applications. There are three main large language model weaknesses to consider when thinking about how to apply them in real-world applications, such as the exemplary applications described herein:

### 1. Inconsistent Accuracy.

Large language models are powerful tools that can provide accurate responses to complex questions. However, despite their impressive capabilities, there is still a risk of inaccurate or false responses, known as “hallucination.”

This phenomenon can have serious implications in critical industries like healthcare and business operations. It is essential to implement safeguards such as human oversight to refine inputs and control outputs to mitigate this risk. Currently, many applications of large language models require human supervision to ensure reliable results but one promising method that aims to fix this is grounding.

### 2. Lack of Enterprise Context.

Large language models have been trained on a vast amount of text data from the internet. Still, they need enterprise-specific context and domain knowledge to provide specific solutions to industry-specific problems. While they can provide general information and context on various topics, they may not have the depth of understanding and experience required to solve complex, industry-specific challenges.

Additionally, language models may not have access to proprietary information or be aware of the specific regulations and policies that govern a particular industry. As a result, they may only sometimes be able to provide accurate or reliable information in the context of a specific enterprise. It is essential to understand these limitations and seek expert advice when dealing with industry-specific issues.

### 3. Limited Controllability.

While language models are powerful and accessible to non-experts, they lack controllability. This means their response to a specific input cannot be easily directed or controlled. The layered approach to building LLMs saves time in training complex systems but limits the ability to control the model's responses in a more demanding environment.

To be effective in a particular setting, LLMs must be part of a larger AI architecture that offers control and fine-tuning through additional training, evaluation, and alternative machine learning approaches.

### 4. Stale Training Data.

Large language models are trained on vast amounts of text data to understand and respond to natural language in a human-like manner. However, their training data is limited to a specific time period and may not reflect the current state of the world. Updating an LLM's knowledge is complex and requires retraining the model, which is extremely expensive.

Instructing the LLM to override certain parts of its knowledge while retaining others is also challenging. Even then, there is no guarantee that the model will not provide outdated information, even if the search engine it is paired with has up-to-date information. This poses a unique challenge in a particular setting where data is often private and constantly changing in real-time.

### 5. Personal Data Risk.

LLMs are trained on vast amounts of text data, including sensitive personal information, which they may have access to while generating responses. This personal information can be leaked through the model's outputs or training data.

Additionally, the training data used to develop LLMs may not always be properly anonymized or secured, which increases the risk of personal data breaches. The use of LLMs in industries handling sensitive personal information, such as healthcare or finance, requires careful consideration and proper security measures to prevent data leakage.

Despite the strengths of LLMs, some challenges must be addressed to fully realize their potential, such as hallucination, stale training data, and a lack of enterprise context.

Artificial neural networks (ANN) first learn from training data and then are later used to make logical inferences from new input data. An input data vector is provided with training data during training sessions and then with new input data when the artificial neural network is used to make inferences. The input data vector is processed with weight data stored in a weighted matrix to create an output data vector.

After processing the input data vector with the weighted matrix, the system creates the output data vector. The output data vector may be combined with an output function to create a final output for the artificial neural network. The output function may be referred to as an activation function. During training sessions, the output data may be compared with a desired target output and the difference between the output data and the desired target output may be used to adjust the weight data within weight matrix to improve the accuracy of the artificial neural network.

Artificial neural networks may comprise many layers of weight matrices such that very complex computational analysis of the input data may be performed. Artificial intelligence relies upon large amounts of very computationally intensive matrix operations to initially learn using training data to adjust the weights in the weight matrices. Later, those adjusted weight matrices are used to perform complex matrix computations with a set of new input data to draw inferences upon the new input data.

LLMs and neural networks can be combined to work together. In some exemplary embodiments, this may be done by using the LLM to generate a set of features that are then fed into the neural network. The neural network can then use these features to make predictions or classifications. For example, in natural language processing, LLMs can be used to generate text features that are then fed into neural networks for tasks such as sentiment analysis, machine translation, and question answering. In computer vision, LLMs can be used to generate image features that are then fed into neural networks for tasks such as object detection, image classification, and scene understanding.

The training of AI includes:

Supervised learning: In supervised learning, the AI is trained on a set of labeled data.

Unsupervised learning: In unsupervised learning, the AI is trained on a set of unlabeled data.

Reinforcement learning: In reinforcement learning, the AI is rewarded for identifying an item correctly. Over time, the AI consistently improves.

The specific approach that is used will depend on the specific needs of the application. For example, if the goal is to identify changes as soon as possible, then supervised learning may be a good option. However, if the goal is to understand the nuances of an item, then unsupervised learning or reinforcement learning may be a better option.

In addition to the type of learning, the training of AI also depends on the size and quality of the data set. A larger data set will typically lead to better performance, but it may also take longer to train the AI. The quality of the data set is also important, as it should be representative of the types of documents that the AI will be used to analyze.

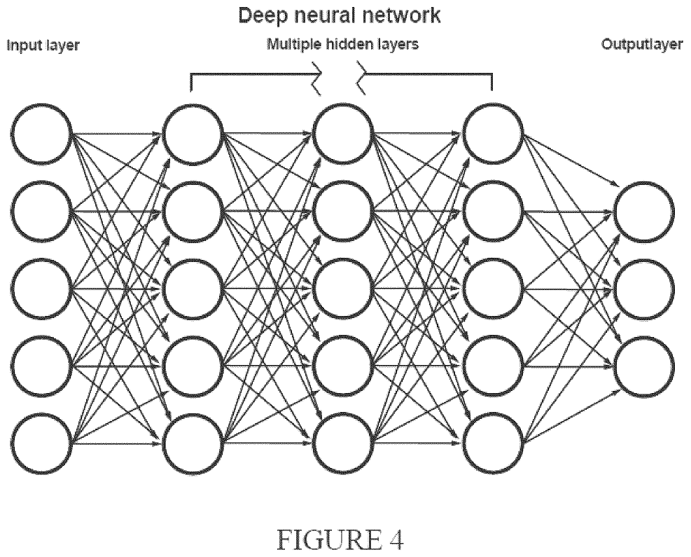

FIG. 4 shows an exemplary deep neural network.

Neural networks, also known as artificial neural networks (ANNs) or simulated neural networks (SNNs), are a subset of machine learning and are at the heart of deep learning algorithms. Their name and structure are inspired by the human brain, mimicking the way that biological neurons signal to one another. Artificial neural networks (ANNs) are comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed along to the next layer of the network.

Neural networks rely on training data to learn and improve their accuracy over time. However, once these learning algorithms are fine-tuned for accuracy, they are powerful tools in computer science and artificial intelligence, allowing one to classify and cluster data at a high velocity. Tasks in speech recognition or image recognition can take minutes versus hours when compared to the manual identification by human experts.

In some exemplary embodiments, one should view each individual node as its own linear regression model, composed of input data, weights, a bias (or threshold), and an output. Once an input layer is determined, weights are assigned. These weights help determine the importance of any given variable, with larger ones contributing more significantly to the output compared to other inputs. All inputs are then multiplied by their respective weights and then summed. Afterward, the output is passed through an activation function, which determines the output. If that output exceeds a given threshold, it “fires” (or activates) the node, passing data to the next layer in the network. This results in the output of one node becoming in the input of the next node. This process of passing data from one layer to the next layer defines this neural network as a feedforward network. Larger weights signify that particular variables are of greater importance to the decision or outcome.

Most deep neural networks are feedforward, meaning they flow in one direction only, from input to output. However, one can also train a model through backpropagation; that is, move in the opposite direction from output to input. Backpropagation allows one to calculate and attribute the error associated with each neuron, allowing one to adjust and fit the parameters of the model(s) appropriately.

In machine learning, backpropagation is an algorithm for training feedforward neural networks. Generalizations of backpropagation exist for other artificial neural networks (ANNs), and for functions generally. These classes of algorithms are all referred to generically as “backpropagation”. In fitting a neural network, backpropagation computes the gradient of the loss function with respect to the weights of the network for a single input-output example, and does so efficiently, unlike a naive direct computation of the gradient with respect to each weight individually. This efficiency makes it feasible to use gradient methods for training multilayer networks, updating weights to minimize loss; gradient descent, or variants such as stochastic gradient descent, are commonly used. The backpropagation algorithm works by computing the gradient of the loss function with respect to each weight by the chain rule, computing the gradient one layer at a time, iterating backward from the last layer to avoid redundant calculations of intermediate terms in the chain rule; this is an example of dynamic programming. The term backpropagation strictly refers only to the algorithm for computing the gradient, not how the gradient is used; however, the term is often used loosely to refer to the entire learning algorithm, including how the gradient is used, such as by stochastic gradient descent. Backpropagation generalizes the gradient computation in the delta rule, which is the single-layer version of backpropagation, and is in turn generalized by automatic differentiation, where backpropagation is a special case of reverse accumulation (or “reverse mode”).

With respect to FIG. 4, according to exemplary embodiments, the system produces an output, which in turn produces an outcome, which in turn produces an input. In some embodiments, the output may become the input.

According to various exemplary embodiments, the burble LLM was and is being trained by burble CEO Alexandra Janelli. Alexandra Janelli stands as a prominent figure in the heart of New York City & Philadelphia's wellness scene, recognized as a leading hypnotherapist and life coach. With over 13 consecutive years of dedicated service, she has earned the respect and trust of a diverse clientele that includes Grammy-winning artists, Academy Award-nominated actors, world-famous photographers, singers, and top executives from various business sectors.

Alexandra's expertise lies in the art of fostering subconscious change, guiding her clients through transformative journeys. Her innovative and effective hypnotherapy techniques have garnered attention and acclaim from major publications such as The New York Times, Crain's, Elle Magazine, Oprah Magazine, Men's Fitness, Forbes, and The New Yorker.

Beyond her professional success, Alexandra is the visionary founder of Modrn Sanctuary, a wellness space that has become synonymous with holistic healing and personal growth. Modrn Sanctuary reflects Alexandra's commitment to providing a sanctuary for individuals seeking positive change in their lives.

Alexandra Janelli's journey in the realm of hypnotherapy, relaxation and subconscious change techniques and life coaching not only impacts the lives of her clients but also contributes to the broader landscape of holistic wellness in the bustling metropolis she calls home. Her passion for helping others create positive, lasting change underscores her influence as a leading figure in the field.

Leveraging the extensive expertise and insights of Alexandra Janelli for training burble's AI represents a significant strategic advantage in the development of a comprehensive and effective wellness platform. Here is how Alexandra's contributions, combined with traditional AI training methods, can enrich burble:

### Rich Content Foundation.

Application: Utilizing Alexandra's writings, recordings, podcasts, radio shows, scripts, case studies, experiences, and programs and the wealth of knowledge shared in her media appearances provides a solid foundation for the content and approach of burble. This ensures that the platform is rooted in proven, effective hypnotherapy and life coaching techniques.

Benefit: Users receive guidance that is both authoritative and authentic, enhancing trust and engagement with the platform.

### Unique Insights and Techniques.

Application: Alexandra's innovative hypnotherapy methods, such as the Breath. Listen. Change. Technique and her ability to foster subconscious change helps in the development of unique features within burble, such as personalized hypnotherapy sessions or guided transformations.

Benefit: Offers users access to specialized wellness interventions that they might not find elsewhere, potentially making burble a leader in digital wellness.

### Credibility and Authority.

Application: Associating burble with Alexandra Janelli, a recognized figure in the wellness community, lends credibility and authority to the platform. This can be particularly valuable in marketing efforts and when building trust with new users.

Benefit: Attracts a wider audience, including those already familiar with Alexandra's work or those seeking reputable sources for their wellness journey.

### Expanding the Training Dataset.

Application: Complementing Alexandra's contributions with publicly available hypnotic research and resources broadens the scope of data used for training burble's AI. This ensures a well-rounded and comprehensive understanding of various hypnotherapy techniques and wellness philosophies.

Benefit: Enhances burbles AI's ability to cater to diverse user needs and preferences, providing a rich, varied content library.

### Visionary Leadership.

Application: Drawing on Alexandra's vision behind Modrn Sanctuary, burble can embody the principles of holistic healing and personal growth, creating an environment that encourages positive change and wellness.

Benefit: Reinforces burble's position as not just an app but a comprehensive wellness platform that reflects a deep commitment to holistic health and personal transformation.

Integrating Alexandra Janelli's depth of knowledge and experience into burble's development ensures that the platform is not only technologically advanced but also deeply connected to the human aspects of wellness and personal growth. This synergy between AI capabilities and human expertise sets burble apart as a pioneering force in the digital wellness space, poised to make a significant impact on the lives of its users.

### Supervised Learning of Burble's LLM.

Burble employs supervised learning, leveraging burble CEO Alexandra Janelli with a powerful AI approach where the model is trained on a labeled dataset. This means that each example in the training set is paired with the correct output. Through this process, the model learns to make predictions or decisions based on new, unseen data, guided by the examples it was trained on.

Here's how supervised training is utilized within burble:

### Personalized Content Recommendations.

Application: By analyzing user interactions and feedback on various sessions (e.g., binaural beats, guided meditations), burble's AI can learn to predict which types of content a user is likely to find beneficial or enjoyable.

Benefit: Enhances the user experience by providing highly targeted recommendations, improving satisfaction and engagement.

### User Behavior Prediction.

Application: Supervised learning algorithms can analyze patterns in user activity (and may with biometric data though wearables and other hardware) to predict future wellness needs or states, such as stress levels.

Benefit: Enables proactive content delivery, offering users the right kind of support exactly when they need it.

### Feedback and Improvement Loop.

Application: By eventually training the AI model on continuous user feedback and engagement metrics, burble can refine its content and features over time, ensuring they align with user preferences and effectiveness.

Benefit: Keeps the app relevant and effective, fostering a deeper connection with its user base.

### Tailored Wellness Programs.

Application: The AI will create customized wellness programs for users by learning from their progress and preferences. For example, if a user consistently engages more with content at certain times of the day or responds well to specific types of encouragement, the AI can adjust their program accordingly.

Benefit: Supports users in achieving their wellness goals more efficiently, enhancing user retention.

### Example of Supervised Training in Burble.

Burble collects data on how users interact with different types of audio sessions, including their ratings, completion rates, and any physiological changes detected via wearable devices during the sessions. This dataset, along with the known outcomes (e.g., improved relaxation, better focus), serves as the basis for training a supervised learning model. The model can then analyze unseen user data to predict which sessions might be most effective for each user, customizing the app's offerings to match individual needs.

### The Burble LLM.

Burble's LLM was meticulously developed from scratch, utilizing the foundational PHP framework as the cornerstone for burble's programming efforts. This approach allowed burble to custom-build burble's backend panel from the very beginning, ensuring a tailored and unique infrastructure. Throughout this process, burble consciously chose not to rely on off-the-shelf tools or pre-existing applications, instead opting to create and name burble's functionalities based on burble's specific requirements and vision. This bespoke development strategy underscores burble's commitment to innovation and provides us with the flexibility to adapt and evolve our platform to meet the changing needs of burble's users.

### Burble's Data Collection.

The EU and the USA have strong laws governing health personal data, and burble must comply with those laws if burble is to use any captured data.

Burble captures a variety of user data. On registration, burble captures a user's language preference, their email, and asks other non-mandatory questions such as the user's age and the user's phone number and address. When a registered user begins with burble, burble captures how often they log in, what inductions they use, what treatments they use, how long they stay on the app, the time they log in, and if they saved any burbles. Burble also saves data across the app, including but not limited to daily burble usage, burble tarot card usage and more.

Burble is designed to collect a variety of user data and use it to enhance personalization and user experience. This approach is quite strategic, as it allows burble to tailor its offerings to the individual needs and preferences of its users, with the intention of improving outcomes related to wellness and engagement with the app.

Here are some potential benefits of the data collection strategy:
1. Language Preference and Basic Information: Capturing basic user information such as language preference, email, age, phone number, and address helps ensure that content is accessible and relevant to each user. Language preference, in particular, enables the delivery of content in the user's preferred language, enhancing usability and comfort.
2. App Usage Metrics: Tracking how often users log in, the types of inductions and treatments they use, session durations, login times, and saved content provides invaluable insights into user behavior and preferences. This data can be used to improve the app's content recommendations, identify popular features, and optimize the user experience based on peak usage times.
3. Engagement with Features: Monitoring specific interactions, such as daily burble usage and tarot card usage, allows for a deeper understanding of which features resonate most with users. This can guide the development of new content and features that align with user interests and needs.
4. Personalization and Adaptation: The comprehensive data collection enables burble to offer a highly personalized experience. By understanding user behavior and preferences, burble can adapt its content dynamically, offering users what they need when they need it, which is crucial for supporting wellness goals effectively.
5. Data-Driven Improvements: The rich dataset collected by burble can be analyzed to continually refine and enhance the app. Insights gained from user interactions can drive content updates, feature enhancements, and overall strategy adjustments to meet evolving user needs.
6. Privacy Considerations: While collecting and utilizing user data offers significant benefits for personalization, it is also essential to manage this data responsibly, ensuring user privacy and compliance with data protection laws such as the General Data Protection Regulation (“GDPR”) and/or California Consumer Privacy Act (“CCPA”). Transparent communication about how data is collected, used, and protected can help build trust with users.

Overall, burble's data collection strategy is poised to create a responsive and engaging wellness platform that aligns closely with individual user needs, potentially setting it apart from less personalized alternatives in the wellness app market.

### Personal Data Protection.

To anonymize personal data that burble collects, ensuring compliance with stringent data protection laws like the EU's General Data Protection Regulation (GDPR) and various privacy laws in the USA, several key strategies may be employed. These methods focus on removing or altering personal identifiers so the data can be used for analysis without compromising user privacy:
1. Data Pseudonymization: This involves replacing private identifiers with fake identifiers or pseudonyms. While the data can still be used for analysis, the pseudonyms prevent direct association with an individual unless additional information is provided. Pseudonymization allows for a balance between utility and privacy, enabling data analysis without exposing personal details.
2. Data Aggregation: By aggregating data, individual records are combined into summaries or statistical measures. For example, rather than analyzing single user behaviors, burble may analyze average usage patterns across thousands of users. Aggregation helps in understanding trends without identifying specific individuals.
3. Randomization: This technique involves adding noise to the data or altering it in a way that the original values cannot be precisely determined. Randomization protects individual data points while still allowing for the overall analysis to be meaningful.
4. Data Masking: This method hides data by obscuring specific data elements within a dataset. For instance, burble could mask parts of an email address or phone number. The data remains usable for certain analyses, even though the masked elements prevent the identification of individuals.
5. Limiting Data Collection: Minimizing the amount of personal data collected to only what is strictly necessary can reduce privacy risks. By focusing on only essential data points for its operations and analyses, burble can ensure greater compliance with privacy laws.
6. Secure Data Storage and Access Controls: Implementing robust security measures for storing anonymized data and strict access controls can further ensure that any data used for analysis is protected against unauthorized access, thereby maintaining user privacy.
7. Regular Audits and Compliance Checks: Regularly reviewing and auditing data management practices can help ensure ongoing compliance with privacy laws. This includes staying updated with changes in legislation and adjusting anonymization practices as needed.

### Integration of Multi-Sensory Elements.

Incorporating binaural beats/music as a backdrop to burble's AI voice audio in burble offers a unique and potentially more immersive wellness experience for burble's users. This combination leverages the therapeutic properties of binaural beats with personalized AI-driven guidance, enhancing the effectiveness of treatments. Burble offers users the option of having no music played along with their custom burble, or one of 2 binaural beats: Alpha Wave beats, Theta Waves or Beta Wave beats, with additional music choices planned. Lastly, burble gives the user the option to end their burble session listing to 5 minutes of their selected audio music should they choose.

Here is how this feature enriches the user experience:

### Enhanced Personalization.

By allowing users to select music to accompany their AI-driven treatments, burble can cater to individual preferences, creating a more personalized and comfortable listening environment. This choice can make the sessions more engaging and enjoyable, potentially increasing the effectiveness of the treatments.

### Synergistic Effects.

Binaural beats influence brainwave patterns, promoting states of relaxation, focus, or meditation. When these are combined with personalized AI voice guidance, the synergistic effect can amplify the benefits of both, offering a powerful tool for stress relief, concentration, or whatever the targeted outcome might be.

### Increased Engagement.

Offering the option to blend binaural beats with treatments can increase user engagement by providing a unique listening experience each time. This novelty can help maintain user interest and commitment to regular use of the app.

### Technical Consideration.

To implement this feature, burble uses audio processing techniques to seamlessly blend the AI-generated voice with the chosen binaural beats without compromising the quality of either. This requires careful control of audio levels, timing, and the tempo to ensure a cohesive and pleasant user experience.

This innovative approach of letting the users to combine binaural beats with AI-generated audio sets burble apart from other wellness apps.

To accomplish this, burble is using FFmpeg. FFmpeg is a free and open-source software project that consists of a suite of libraries and programs for handling video, audio, and other multimedia files and streams. FFmpeg is a powerful and versatile tool that supports a wide range of audio formats, making it ideal for processing and manipulating audio files in various ways.

Burble installed the FFmpeg library on burble's server.

Here is how FFmpeg supports the specific needs of burble:

### 1. Combining Audio Tracks.

FFmpeg seamlessly merges the binaural beats music track with the AI-generated voice track. This is achieved through the use of FFmpeg commands that mix multiple audio sources into a single output file, ensuring the voice and music blend smoothly without compromising the integrity of either.

### 2. Adjusting Audio Levels.

To ensure that neither the binaural beats nor the AI voice dominates the audio experience, FFmpeg is used to adjust the volume levels of each track before merging. This balance is crucial for maintaining the effectiveness of the binaural beats while ensuring the AI voice remains clear and understandable.

### 3. Converting Audio Formats.

Burble on occasion may require audio content to be in specific formats for optimal compatibility and performance across different devices. FFmpeg offers extensive capabilities for converting audio files between various formats, ensuring users have a consistent experience regardless of their device.

### 4. Optimizing for Streaming.

For an app like burble, it is essential that audio files are optimized for streaming to minimize buffering and lag. FFmpeg optimizes the audio files for streaming by adjusting bit rates and using codecs that are designed for efficient transmission over the internet.

### 5. Batch Processing.

FFmpeg supports batch processing, allowing for the automation of audio editing tasks on multiple files, saving time and ensuring consistency across the audio library.

By leveraging FFmpeg's robust features, the burble development team has created a rich, immersive audio experience for users, enhancing the personalization and effectiveness of the wellness sessions offered by the app.

How it is done:

Burble set-up FFmpeg on burble's server to enable the combination of AI-generated narratives with pre-recorded binaural beats music chosen by our users. Other embodiments might not employ AI.

Here is how burble makes it happen:

First, burble retrieves the narrative content produced by burble's AI. This content is then transformed into an audio format. Following this, burble incorporates a user's music selection into the process. Using specialized FFmpeg commands, burble blends the AI's audio narrative with the chosen music tracks into a single, cohesive audio file.

Step by step:

Step One: Users pick their preferred background tracks to accompany their personalized burble session.

Step Two: Burble's custom coding transforms the AI's written stories into spoken audio with the help of FFmpeg.

Step Three: Burble then blended this AI-generated audio with the selected music tracks, creating a unified audio experience.

Step Four: The final, mixed audio file is uploaded to burble's server, ready for the user to enjoy.

Through this process, each burble session becomes a custom-crafted audio journey, perfectly aligned with the user's preferences and enhancing their experience.

In illustrative form:

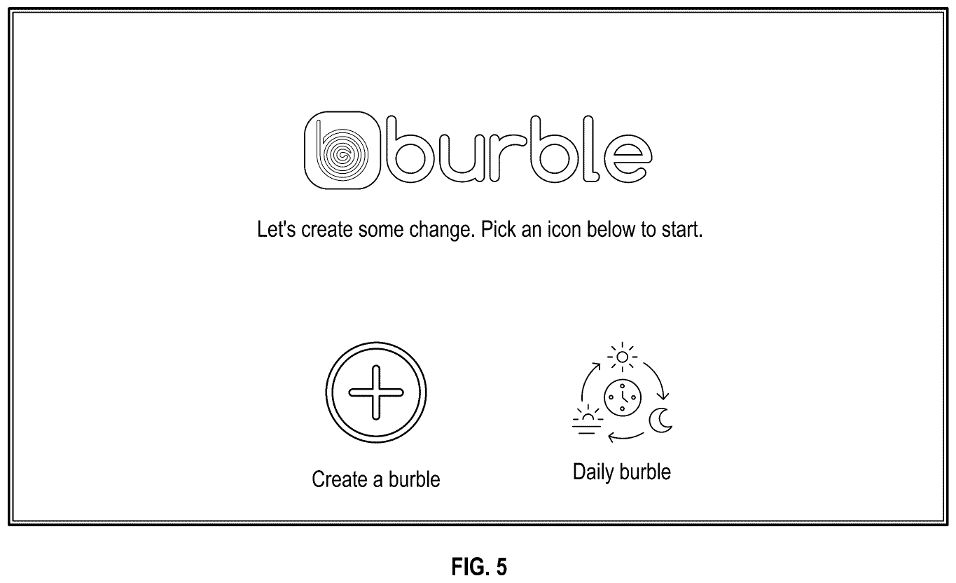

FIG. 5 Shows an exemplary screenshot for initiating the burble process.

According to exemplary embodiments, a custom curated recording may be considered a burble. A burble may be anything that combines and/or creates a custom adventure using media and/or multimedia. Also shown in FIG. 5 is a selection for a daily burble. A daily burble has AI created content to help users throughout the day by asking a variety of questions and providing perspective shifting processes and practices. Insight cards with write-ups that are unique to the daily burble helps user awareness through activities.

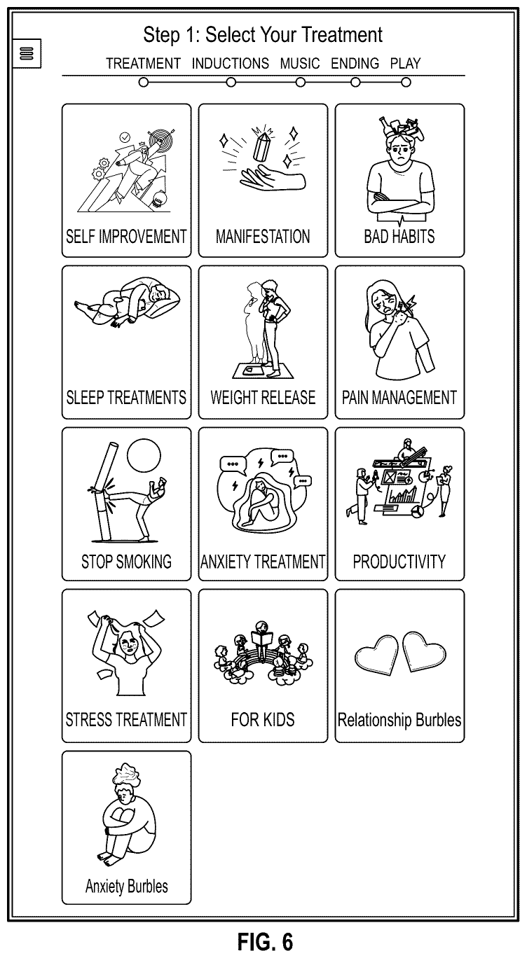

FIG. 6 Shows an exemplary screenshot for selecting a treatment in the burble process. Here, a user selected self-improvement.

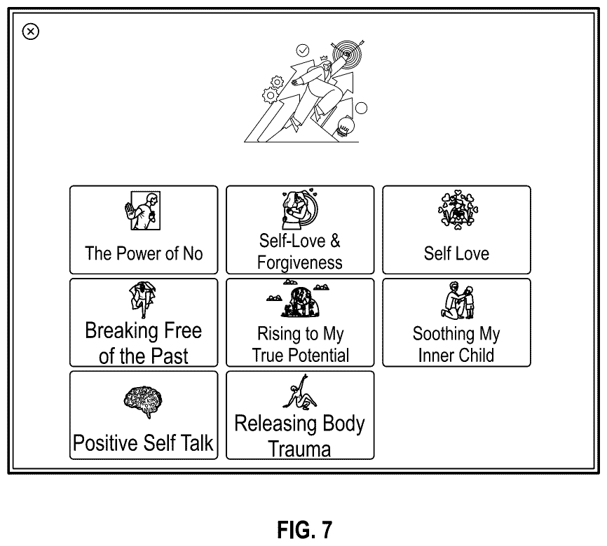

FIG. 7 Shows an exemplary screenshot for selecting an area to focus on within the treatment in the burble process.

Here, the user selected positive self-talk.

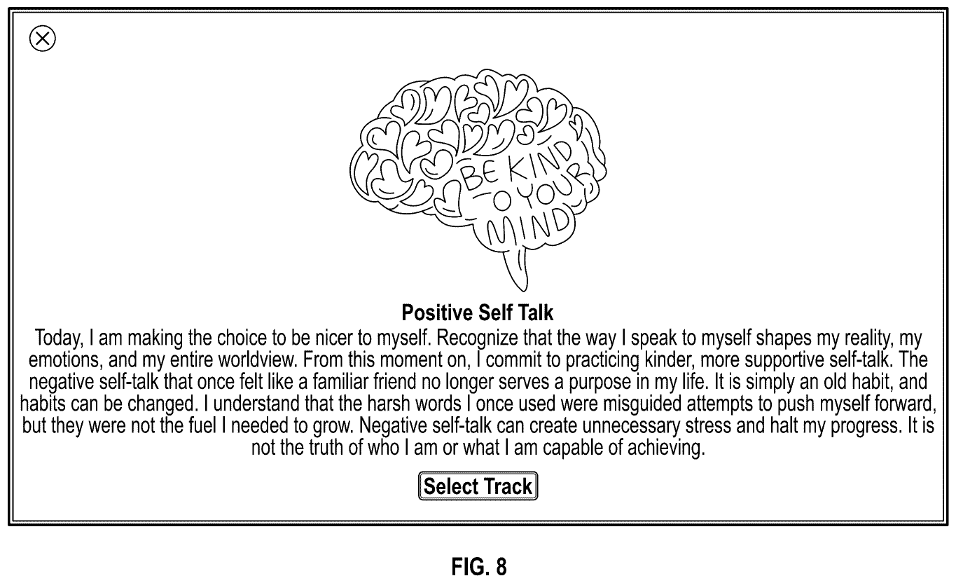

FIG. 8 Shows an exemplary screenshot for selecting a track in the area to focus on within the treatment in the burble process.

In addition to being presented with information about positive self-talk, here, the user selected “Select Track.”

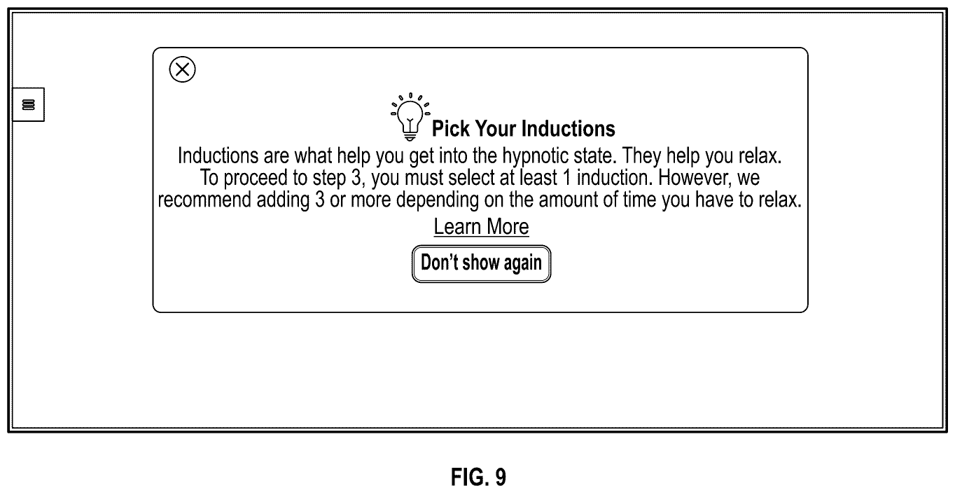

FIG. 9 Shows an exemplary screenshot for selecting an induction in the area to focus on within the treatment in the burble process.

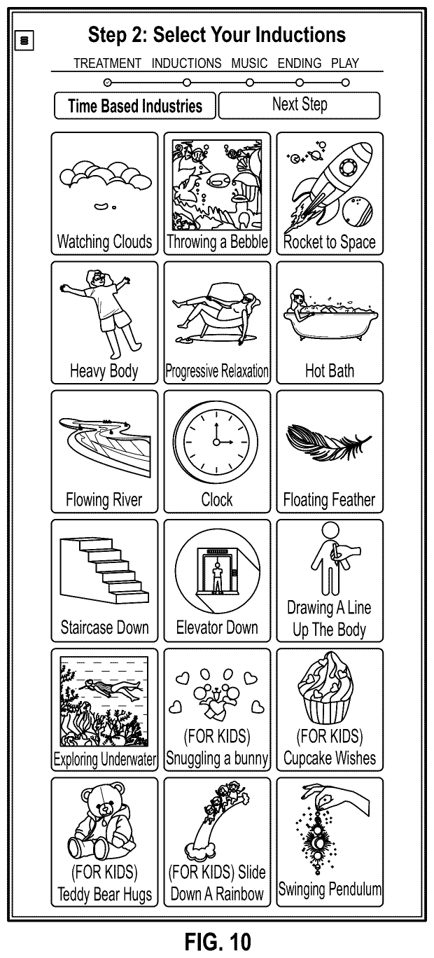

FIG. 10 Shows another exemplary screenshot for selecting an induction in the area to focus on within the treatment in the burble process.

Here, the user selected “Rocket to Space.”

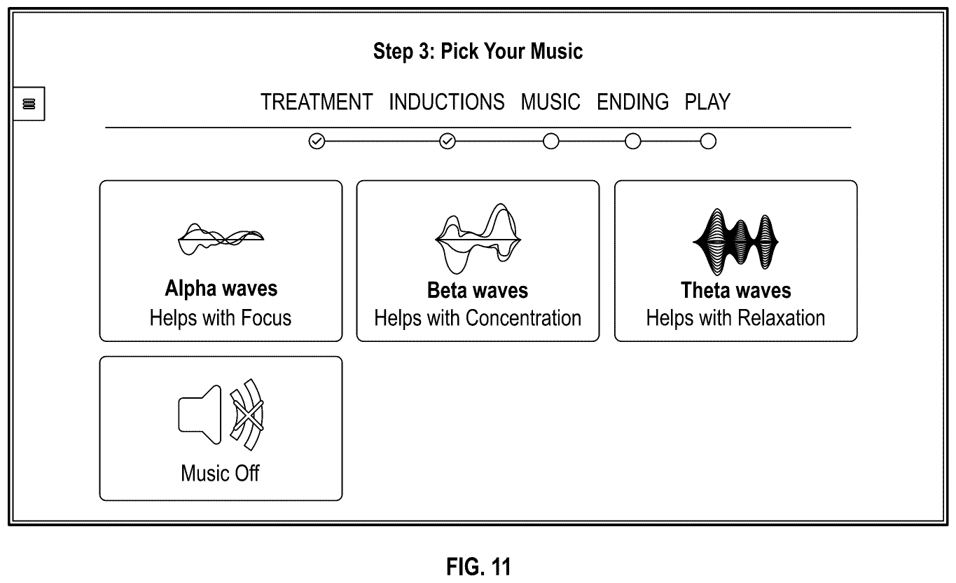

FIG. 11 Shows an exemplary screenshot for selecting music in the burble process.

Here, the user selected Beta waves.

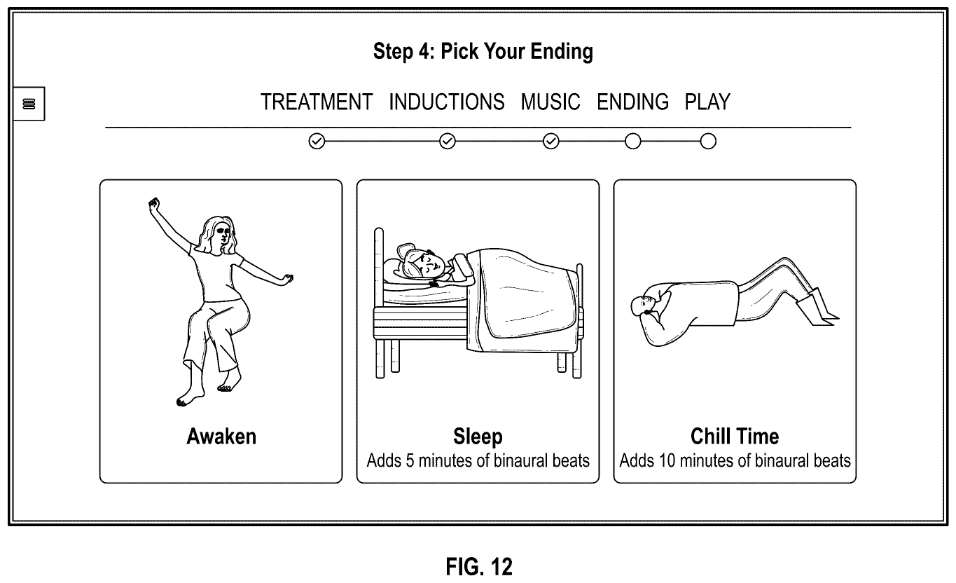

FIG. 12 Shows an exemplary screenshot for selecting an ending in the burble process.

Here, the user selected awaken.

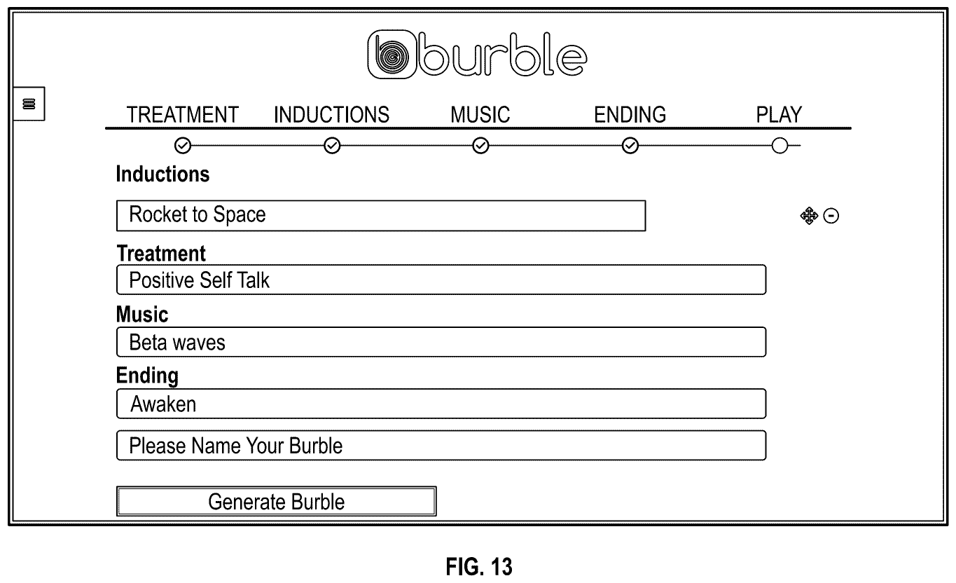

FIG. 13 Shows an exemplary screenshot for generating a burble in the burble process.

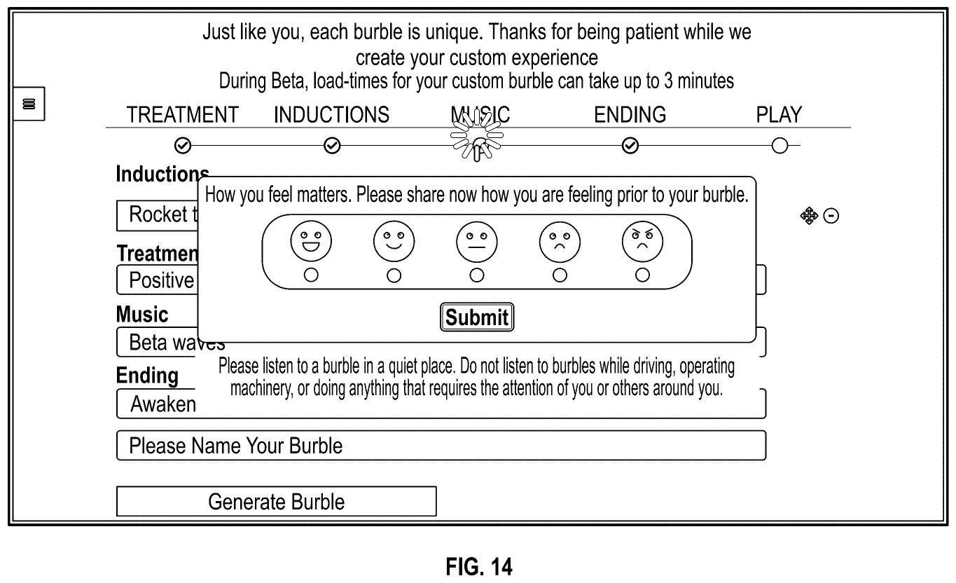

FIG. 14 Shows an exemplary screenshot for providing user feedback while the burble is generating in the burble process.

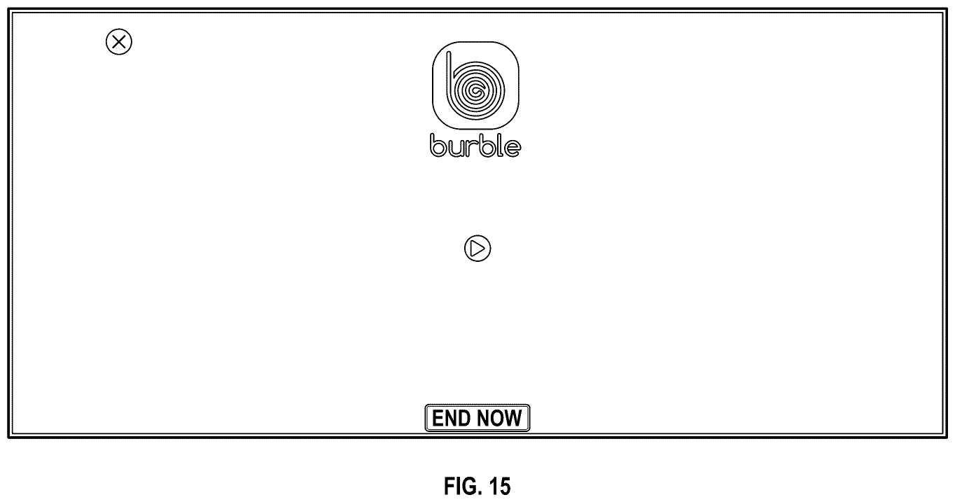

FIG. 15 Shows an exemplary screenshot for playing a burble in the burble process.

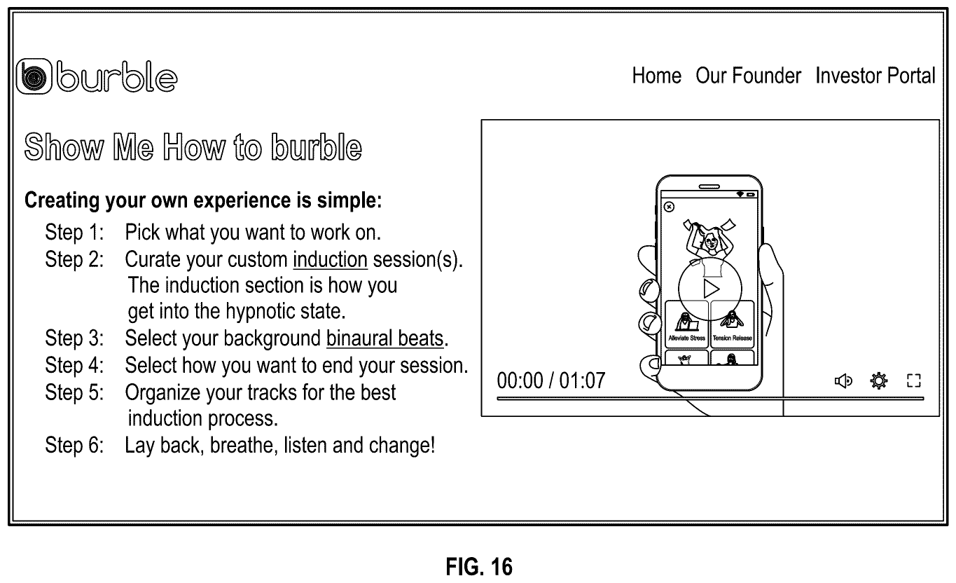

FIG. 16 Shows an exemplary screenshot for an overview of the burble process.

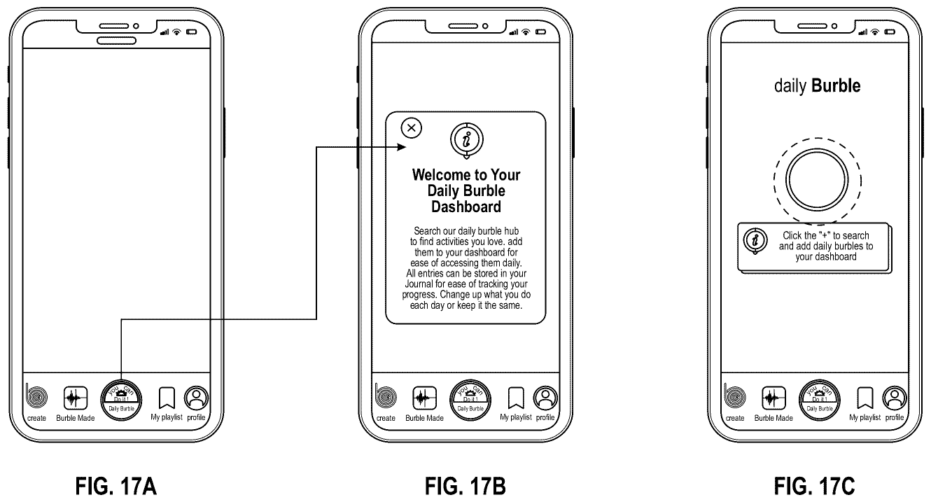

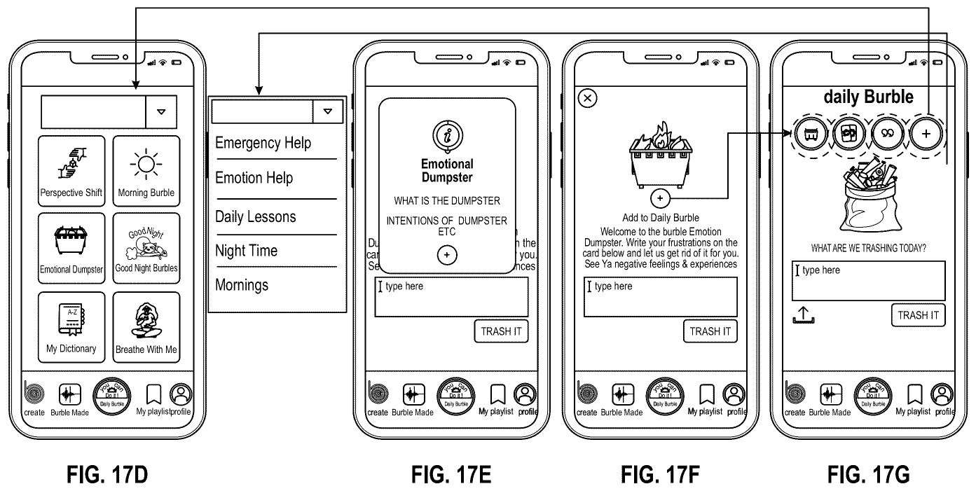

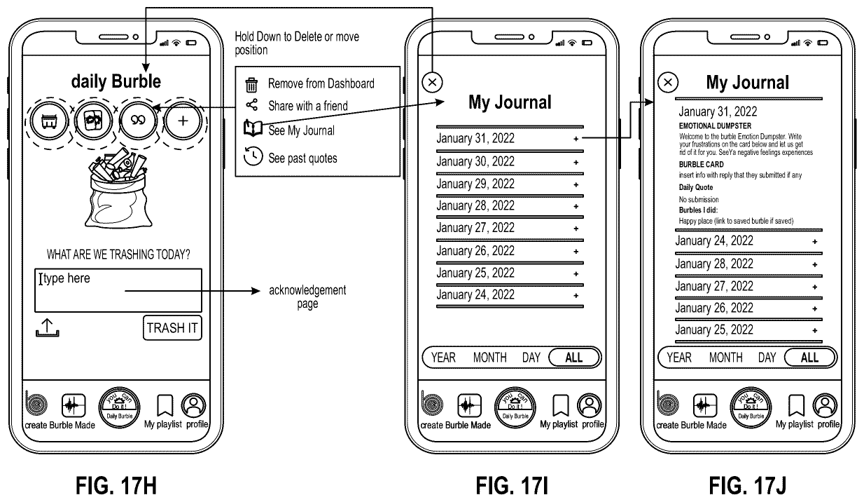

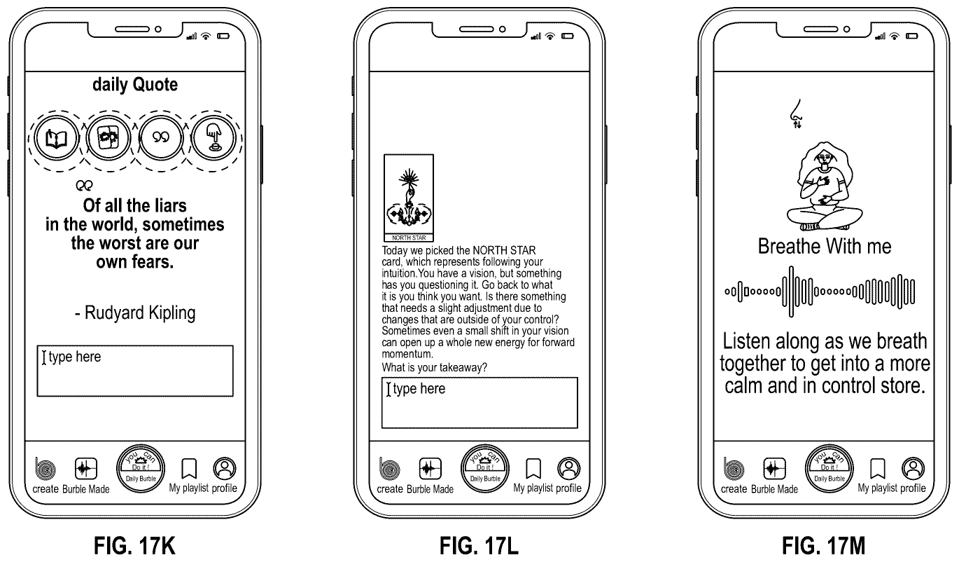

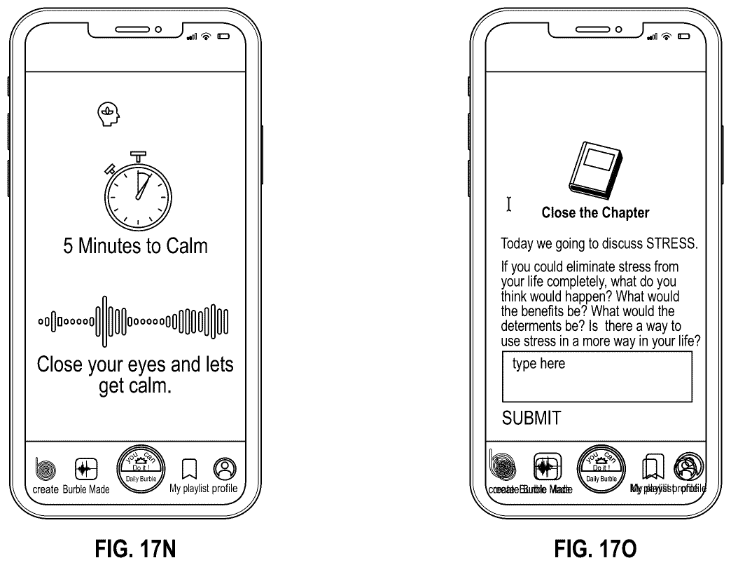

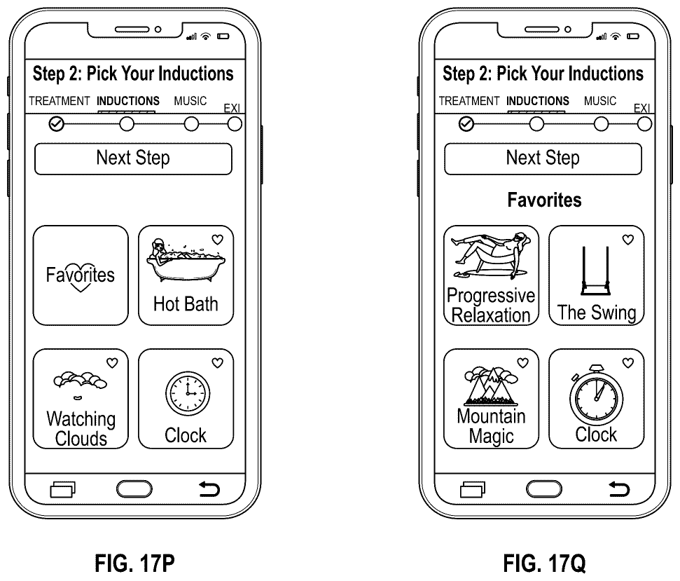

FIGS. 17A-17Q Show a customizable dashboard for burble.

As shown in FIGS. 17A-17Q, there is a customizable dashboard for users to pick their favorite inductions, treatments, daily burble activities, etc. for quick access. The burble home screen dashboard comes pre-loaded with two icons that will bring you to the Daily burble or to create a custom burble. Under the Daily burble section the user can select from an array of activities and also create a custom dashboard of their favorites for easy access, as shown in FIGS. 17A-17Q. For example, if the user wanted to add the Emotional Dumpster to their Daily burble dashboard for easy access they would simply click the add to dashboard icon, where it would then be added to the dashboard and shown for easy access when in other activities. In addition, in the custom burble section, the user will be able to have a quick reference to their favorite inductions, treatments, music and endings as shown in FIGS. 17A-17Q.

### Algorithms.

For burble, leveraging data mining algorithms significantly enhances burble's ability to deliver personalized wellness experiences.

Here are three examples of data mining algorithms that may be effectively utilized:

### 1. Clustering Algorithm for User Segmentation.

Description: Clustering algorithms, such as K-means, can be used to segment burble users into different groups based on their usage patterns, preferences, and wellness goals. By analyzing data points like the types of content users interact with, their activity levels, and preferred times for app usage, burble can identify distinct user segments.

Application: This segmentation allows for the tailored creation of wellness content and recommendations for each group, improving personalization and user engagement. For example, a cluster of users who frequently engage with stress management content might receive more targeted interventions in this area such as a comedy burble and/or an anonymous group meeting where users can be in a scheduled group setting, anonymous, all talking about anxiety and supporting each other.

### 2. Association Rule Mining for Personalized Content Recommendations.

Description: Association rule mining, utilizing algorithms like Apriori or FP-Growth, can discover interesting associations and correlations between different types of content and user actions within the burble app. This method identifies patterns such as “Users who engage with morning meditation sessions also prefer relaxation music in the evening.”

Application: These insights can inform the development of highly personalized content recommendations, suggesting new wellness activities to users based on their existing preferences and behaviors. It can enhance the user experience by exposing them to new, relevant content they are likely to enjoy and benefit from.

### 3. Decision Trees for Predictive Modeling.

Description: Decision trees can be used to predict future user behaviors based on historical data. By creating a model that considers various factors, including user engagement metrics, feedback, and biometric data collected through wearables, decision trees can help predict user needs and wellness states.

Application: Predictive modeling can proactively offer specific wellness interventions. For instance, if the model predicts that a user is likely to experience increased stress levels based on their historical data and current biometric feedback, burble could automatically suggest stress-reduction sessions before the user even recognizes the need.

Implementing these data mining algorithms requires careful consideration of privacy and ethical use of data, ensuring that all user data is anonymized and securely handled. By applying these algorithms, burble can enhance its AI-driven platform, offering more personalized, timely, and effective wellness interventions, thereby improving overall user satisfaction and engagement with the app.

Burble may use a combined approach that incorporates clustering, association rule mining, and decision trees for data analysis using python. Python can be used due to its robust libraries for data science such as scikit-learn for machine learning models, and pandas for data manipulation. Integrating these methods effectively requires a nuanced understanding of the data and the specific goals of the analysis.

Burble's Neural Network.

Neural network-LLM work with natural language processing, however a neural network will take things and get better inferences, using/based on different types of input (feedback and how it weighs this thing against that thing). Burble is planning a neural network to enhance its capability to deliver highly personalized and adaptive wellness content.

Here is how burble plans to use a neural network:

### 1. Understanding User Preferences and Behavior.

The burble neural network may analyze vast amounts of user interaction data (such as choices made within the app, feedback on sessions, time spent on various activities) to learn and predict what types of content each user prefers or is likely to find beneficial.

### 2. Personalizing Content.

Based on its learning, the burble neural network can tailor the wellness content for each user. For example, if a user frequently engages with stress reduction sessions and rates them highly, the neural network will learn to recommend similar or complementary sessions, maybe even before the user realizes they need them. For example, burble may “watch” how often a user chooses a treatment, and if the user finishes the treatment. Burble may “watch” if a user saves a treatment, and goes back to use the treatment again. If they do, burble may “watch” how often they replay a treatment. Burble may collect simple feedback at the end of each treatment.

In other exemplary embodiments, burble may employ an AI chatbot (“AI”) asking questions of a user like:
AI: What's troubling you today?
Answer: I can't sleep.
AI: What is causing you not to be able to sleep?
Answer: My mind is too active worrying.
AI: What is worrying you the most?
Answer: My finances and making ends meet.

Burble has learned in this simple example that the person is not sleeping because they are worried about money.

Burble is taking all of that usage data, and “learning” what all the users are favoring. Using this data, burble can take what seems like unrelated topics and, using a neural network, offer deeper treatments.

### 3. Adapting to Changes Over Time.

People's needs and preferences change over time, and the burble neural network may be capable of adapting to these changes. As the burble neural network continuously analyzes new data from user interactions, it may update its understanding of what each user prefers, ensuring the content remains relevant and engaging.

### 4. Enhancing Predictive Models.

With the integration of wearable device data, such as heart rate or activity levels, a neural network can also predict when a user might need a specific type of wellness intervention. For instance, detecting patterns that precede a stress response could trigger timely suggestions for preemptive relaxation exercises.

### 5. Providing Insights for Content Development.

By analyzing which types of content are most and least effective across different user segments, the burble neural network can provide insights to the burble team about gaps in their content offering or emerging wellness trends they might capitalize on.

In essence, the burble neural network may allow burble to become a smart, learning system that not only personalizes content in real-time but also evolves with its users, making wellness support more effective and engaging. This approach leverages the power of AI to meet the unique needs of each user, providing a tailored experience that can help more effectively manage stress, improve mental health, and enhance overall well-being.

### Burble Chatbot.

Burble believes a chatbot could significantly enhance the user experience on burble by providing interactive, personalized support and guidance.

For example:

### 1. User Onboarding and Profile Setup Wizard.

Function: This wizard is designed to smoothly guide new users through their initial setup, gathering their preferences and specific wellness objectives. Utilizing a conversational approach, the AI delves into the users' concerns with targeted questions, employing logical structures such as “if/then” scenarios to unearth the root causes of their issues. This process enables burble to craft a fully personalized program that addresses both the superficial and deeper underlying problems for a holistic treatment approach.

For instance, if a user expresses difficulties with sleep, the AI chatbot might inquire, “If you're finding it hard to sleep, can you identify a primary cause? Is it 1. Work, 2. Friendships, 3. Money/Finances, or 4. General dissatisfaction?” Upon selecting “Money/Finances,” the AI acknowledges the stress financial worries can cause and probes further to uncover additional concerns that might be affecting sleep quality. This conversation continues until the AI identifies potential root causes of the sleep disturbance.

Leveraging the insights gained, the AI then assembles a tailored program that might include sessions like “Transform Your Financial Mindset,” alongside “Guided Sessions for Sleep” and “Techniques to Calm Racing Thoughts,” offering a comprehensive solution aimed at the user's specific sleep issues rather than a one-size-fits-all remedy.

Benefit: This interactive and personalized onboarding process not only makes the initial setup more engaging but also ensures that the content and recommendations provided by burble are finely tuned to meet the individual needs of each user right from the start, setting the stage for a more effective and satisfying wellness journey.

### 2. Daily Check-ins and Reminders.

Function: Send users daily prompts to check in on their mental state or remind them to complete wellness activities.

Benefit: Increases user engagement and adherence to wellness routines.

### 3. Personalized Recommendations.

Function: Suggest specific content based on the user's mood, recent activities, or goals, using input from the user's interactions with the chatbot.

Benefit: Delivers a highly personalized experience, potentially improving wellness outcomes.

### 4. FAQs and Support.

Function: Answer common questions about using burble, troubleshooting issues, or navigating the app.

Benefit: Provides instant support, enhancing user satisfaction and reducing frustration.

### 5. Feedback Collection.

Function: Ask users for feedback on sessions they have completed, using natural language processing to understand the responses.

Benefit: Gathers valuable insights to improve content and app functionality.

### 6. Mindfulness and Breathing Exercises.

Function: Lead users through guided mindfulness or breathing exercises directly within the chat interface.

Benefit: Offers immediate stress relief options and introduces users to activities they might not have explored.

### 7. Crisis Management.

Function: Recognize when a user might be in distress and provide resources or encourage them to seek professional help.

Benefit: Adds a layer of safety, showing users that burble cares about their well-being.

### 8. Community Engagement.

Function: Facilitate connections with a wider community by suggesting group challenges or connecting users with similar goals.

Benefit: Builds a sense of community and belonging, which can be crucial for motivation and support.

### 9. Learning and Adaptation.

Function: Continuously learn from interactions to improve its responses and recommendations over time.

Benefit: Ensures the chatbot becomes more effective the more a user interacts with it, enhancing the personalized experience.

Integrating a chatbot into burble will significantly enrich the user experience by providing real-time, interactive support that is tailored to individual needs, thereby fostering a more engaging and supportive wellness journey.

Given the innovative approach of burble, particularly its use of AI for generating personalized content and potentially integrating with wearable technology for real-time user data analysis, it is feasible to consider that such advancements could indeed contribute to improvements in computer functionality, particularly in the context of app performance and efficiency.

Here are a few ways burble might influence improvements:

### 1. Efficient Data Processing.

How: By leveraging AI algorithms optimized for performance, burble can process large datasets (e.g., user preferences, biometric data) more efficiently.

Impact: This can lead to faster processing speeds, allowing for quicker generation of personalized content and recommendations without significant delays.

### 2. Reduced Storage Requirements.

How: Ai-driven content generation means burble doesn't need to store vast amounts of pre-created content. Instead, it can generate content on-the-fly based on user data.

Impact: This approach can significantly reduce the storage requirements for the app, freeing up space on servers and potentially reducing costs associated with data storage.

### 3. Improved Latency.

How: By optimizing AI models and utilizing edge computing principles (processing data closer to the source of data), burble can minimize the latency experienced during data transmission and processing.

Impact: Users can enjoy a smoother, more responsive experience with minimal lag, enhancing overall satisfaction with the app.

### 4. Scalability.

How: AI and cloud technologies enable burble to scale its services dynamically based on user demand without the need for manual intervention.

Impact: This flexibility ensures that burble can manage peak loads efficiently, maintaining performance without degradation even as the user base grows.

### 5. Energy Efficiency.

How: Advanced Ai models can be designed to be energy-efficient, requiring less computational power to generate and deliver personalized content.

Impact: This not only reduces the energy footprint of running burble but can also extend the battery life of mobile devices using the app, contributing to a better user experience.

In summary, through the strategic use of Ai and modern computing technologies, burble has the potential to positively impact aspects such as storage requirements, processing speed, latency, scalability, and energy efficiency. These improvements not only enhance the performance and user experience of burble itself but could also set new standards for the development and optimization of similar applications in the future.

1. A computer-implemented method of training a neural network and a large language model for automatically collecting, analyzing, and transmitting data to induce a mental state and change a behavior in a mammal, the method comprising:
collecting a first set of data relevant to automatically collecting, analyzing, and transmitting data to induce the mental state and change a behavior in a mammal;
applying one or more transformations to the collected first set of data to create a first modified set of data;
creating a first training set comprising the first collected set of data, the first modified set of data and a first set of non-transformed data;
training the neural network and large language model in a first stage using the first training set;
creating a second training set for a second stage of training comprising the first training set and the first set of non-transformed data that are incorrectly transformed after the first stage of training; and
training the neural network and large language model in a second stage using the second training set to automatically collect, analyze, and transmit data to induce a mental state and change a behavior in a mammal.

2. The computer-implemented method of training a neural network and a large language model for automatically collecting, analyzing, and transmitting data to induce a mental state and change a behavior in a mammal of claim 1, the method further comprising:
collecting the first set of data including data from a wearable device.

3. The computer-implemented method of training a neural network and a large language model for automatically collecting, analyzing, and transmitting data to induce a mental state and change a behavior in a mammal of claim 1, the method further comprising:
collecting the first set of data including data from a keyboard, keypad, or a mobile device touchscreen.

4. The computer-implemented method of training a neural network and a large language model for automatically collecting, analyzing, and transmitting data to induce a mental state and change a behavior in a mammal of claim 1, the method further comprising:
collecting the first set of data including a heart rate.

5. The computer-implemented method of training a neural network and a large language model for automatically collecting, analyzing, and transmitting data to induce a mental state and change a behavior in a mammal of claim 1, the method further comprising:
collecting the first set of data including steps taken.

6. The computer-implemented method of training a neural network and a large language model for automatically collecting, analyzing, and transmitting data to induce a mental state and change a behavior in a mammal of claim 1, the method further comprising:
collecting the first set of data including blood oxygen saturation.

7. The computer-implemented method of training a neural network and a large language model for automatically collecting, analyzing, and transmitting data to induce a mental state and change a behavior in a mammal of claim 1, the method further comprising:
collecting the first set of data including heart rate variability.

8. The computer-implemented method of training a neural network and a large language model for automatically collecting, analyzing, and transmitting data to induce a mental state and change a behavior in a mammal of claim 1, the method further comprising:
collecting the first set of data including systolic and diastolic blood pressure.

9. The computer-implemented method of training a neural network and a large language model for automatically collecting, analyzing, and transmitting data to induce a mental state and change a behavior in a mammal of claim 1, the method further comprising:
collecting the first set of data including a blood sugar level.

10. The computer-implemented method of training a neural network and a large language model for automatically collecting, analyzing, and transmitting data to induce a mental state and change a behavior in a mammal of claim 1, the method further comprising:
collecting the first set of data including a body mass index.

11. The computer-implemented method of training a neural network and a large language model for automatically collecting, analyzing, and transmitting data to induce a mental state and change a behavior in a mammal of claim 1, the method further comprising:
collecting the first set of data including bone density.

12. The computer-implemented method of training a neural network and a large language model for automatically collecting, analyzing, and transmitting data to induce a mental state and change a behavior in a mammal of claim 1, the method further comprising:
collecting the first set of data including electrical activity of a heart.

13. The computer-implemented method of training a neural network and a large language model for automatically collecting, analyzing, and transmitting data to induce a mental state and change a behavior in a mammal of claim 1, the method further comprising:
collecting the first set of data including a perfusion index.

14. The computer-implemented method of training a neural network and a large language model for automatically collecting, analyzing, and transmitting data to induce a mental state and change a behavior in a mammal of claim 1, the method further comprising:
collecting the first set of data including body temperature.

15. The computer-implemented method of training a neural network and a large language model for automatically collecting, analyzing, and transmitting data to induce a mental state and change a behavior in a mammal of claim 1, the method further comprising:
collecting the first set of data including sleep duration.

16. The computer-implemented method of training a neural network and a large language model for automatically collecting, analyzing, and transmitting data to induce a mental state and change a behavior in a mammal of claim 1, the method further comprising:
collecting the first set of data including a sleep stage.

17. The computer-implemented method of training a neural network and a large language model for automatically collecting, analyzing, and transmitting data to induce a mental state and change a behavior in a mammal of claim 1, the method further comprising:
collecting the first set of data including brain wave activity.

18. The computer-implemented method of training a neural network and a large language model for automatically collecting, analyzing, and transmitting data to induce a mental state and change a behavior in a mammal of claim 1, the method further comprising:
collecting the first set of data including a finger print.

19. The computer-implemented method of training a neural network and a large language model for automatically collecting, analyzing, and transmitting data to induce a mental state and change a behavior in a mammal of claim 1, the method further comprising:
collecting the first set of data from a child.

20. The computer-implemented method of training a neural network and a large language model for automatically collecting, analyzing, and transmitting data to induce a mental state and change a behavior in a mammal of claim 1, the method further comprising:
collecting the first set of data from an adult human.
